(19) United States
(12) Patent Application Publication
(10) Pub. No.: US 2014/0107104 A1
(43) Pub. Date: Apr. 17, 2014

(54) COMBINATION MEDICINE FOR TREATMENT OF DEPRESSION

(71) Applicants: Akinori Nishi, Fukuoka (JP); Katsunori Kobayashi, Tokyo (JP); Tsuyoshi Miyakawa, Aichi (JP); Paul Greengard, New York, NY (US)

(72) Inventors: Akinori Nishi, Fukuoka (JP); Katsunori Kobayashi, Tokyo (JP); Tsuyoshi Miyakawa, Aichi (JP); Paul Greengard, New York, NY (US)

(21) Appl. No.: 14/006,172

(22) PCT Filed: Mar. 22, 2012
(86) PCT No.: PCT/JP2012/001996

§ 371 Date: Dec. 4, 2013

Related U.S. Application Data
(60) Provisional application No. 61/466,157, filed on Mar. 22, 2011.

(51) Int. Cl. A61K 31/55; G01N 33/50; C12Q 1/68; A61K 31/135

(52) U.S. Cl. CPC A61K 31/55; A61K 31/135; G01N 33/5023; C12Q 1/68
USPC 514/217; 514/651; 514/217.02; 435/6.12; 506/9

(57) ABSTRACT

The present invention provides a combination medicine for treatment of depression, comprising a combination of (A1) an antidepressant and either (B1) a dopamine D1 receptor agonist or (C1) a dopamine D1 receptor antagonist; and a method for screening for an antidepressant that in combination with a dopamine D1 receptor agonist provides an improvement in treatment of depression, the method comprising the steps of: administering, to a mammal, (A2) a compound having an antidepressant action and (B1) a dopamine D1 receptor agonist, and detecting a greater increase in depression-related gene expression, dopamine D1 receptor expression and/or dopamine D1 receptor signaling in comparison with the case where (A2) the compound having an antidepressant action or (B1) the dopamine D1 receptor agonist is administered.

### TECHNICAL FIELD

The present invention relates to antidepressive medication, for example, a combination medicine for treatment of depression, a method for treatment of depression, a method for administration for treatment of depression, use of an antidepressant and a dopamine D1 receptor agonist for the manufacture of medicine for treatment of depression, a method for screening for an antidepressant that in combination with a dopamine D1 receptor agonist provides an improvement in treatment of depression, a method for screening for a dopamine D1 receptor agonist that in combination with an antidepressant provides an improvement in treatment of depression, a kit for screening for an antidepressant that in combination with a dopamine D1 receptor agonist provides an improvement in the treatment of depression, and a kit for screening fora dopamine D1 receptor agonist that in combination with an antidepressant provides an improvement in treatment of depression.

### BACKGROUND ART

For treating depression, antidepressants, such as a tricyclic antidepressant, a tetracyclic antidepressant, a selective serotonin reuptake inhibitor (SSRI), and a selective serotonin and/or noradrenaline reuptake inhibitor (SNRI) are used. Although such medication improves 70 to 80% of depression cases, these medicines have problems including the following: the medicines generally require 2 or 3 weeks of continued administration before the effects thereof are produced; refractory depression that does not respond to the medicines accounts for 20 to 30% of all the depression cases; and the rate of recurrence after improvement of depressive phase is rather high. Therefore, these antidepressants are not satisfactory, and improved antidepressive medication by, for example, combinational use with other medicines has been desired.

Regarding other medicines used together with an antidepressant, methylphenidate, which is used for treating depression, is an indirect agonist that promotes dopamine release. However, methylphenidate does not have receptor specificity and causes dependence as a social issue. Dopamine receptor agonists known as an antiparkinson drug are reported to be effective as a medicine for treating refractory depression, manic depression, or the like (Non Patent Literature 1). Many of the dopamine receptor agonists are medicines mainly targeting dopamine D2 receptor, and for example, it is known that an atypical antipsychotic, which exhibits dopamine D2 receptor antagonism when used with an antidepressant, is effective on manic depression (Non Patent Literature 2). In addition, a method for treatment of depression or anxiety by administering, to a mammal, a dopamine D3 receptor agonist and a monoamine (for example, serotonin, dopamine, norepinephrine) reuptake inhibitor is disclosed (Patent Literature 1), but a further improved combination medicine and treatment method, etc., using a dopamine receptor agonist together with an antidepressant, have been desired.

In patients with depression, atrophy of the hippocampus caused by stress or the like has been confirmed (Non Patent Literature 3). The hippocampus includes regions of CA1 to CA3. The dentate gyrus which is included in the hippocampus in a broad concept has a layer of cells called granule cells, and the granule cells extend their axons toward the inside of the CA3 region of the hippocampus. Although most of neuronal population in the adult mouse dentate gyrus is composed of mature granule cells, the increase in the proportion of relatively young granule cells resulting from accelerated neurogenesis seems to alter the functional role of the dentate gyrus in the hippocampal circuit.

Kobayashi et al. revealed that chronic administration of an antidepressant to a mouse returns the mature neurons in the hippocampal dentate gyrus to their premature state (hereafter sometimes referred to as “dematured dentate gyrus”) and reported the change as “dematured dentate gyrus induced by chronic administration of antidepressant” (Non Patent Literature 4). Since dematuration greatly alters the functional properties of granule cells and is induced in the entire granule cells, induction of dematuration of the hippocampal dentate gyrus is thought to be important in the action mechanism of an antidepressant. Therefore, focusing attention on the above, the inventors started the development of the present invention in order to find out further treatment of depression.

### CITATION LIST

### Patent Literature

### [PTL 1]

JP 2002-370976 A

### Non Patent Literature

### [NPL 1]

Arch Gen Psychiatry. 2007, 6: 327-337

### [NPL 2]

N Engl J Med. 2011, 364: 51-59

### [NPL 3]

J Psychiatry Neurosci. 2010, 35(5): 337-343,

### [NPL 4]

Proc Natl Acad Sci USA. 2010, 107: 8434-8439

### SUMMARY OF INVENTION

### Technical Problem

As described above, in the treatment of depression under present circumstances, medicines targeting dopamine D1 receptor are not used, and Patent Literature 1 does not make any reference to combination use of a dopamine D1 receptor agonist and an antidepressant. In addition, under present circumstances, no dopamine D1 receptor agonists effective, based on the above finding of “dematured dentate gyrus” induced by chronic administration of an antidepressant to a mouse, in the treatment of depression when used together with an antidepressant have been found out.

Then, an object of the present invention is to provide a combination medicine for treatment of depression, a pharmaceutical composition, a method for treatment of depression, and a method for administration for treatment of depression, in all of which an antidepressant and a dopamine D1 receptor are used; use of an antidepressant and a dopamine D1 receptor agonist for the manufacture of medicine for treatment of depression; a method for screening for an antidepressant that in combination with a dopamine D1 receptor agonist provides an improvement in treatment of depression; a method for screening for a dopamine D1 receptor agonist that in combination with an antidepressant provides an improvement in treatment of depression; a kit for screening for an antidepressant that in combination with a dopamine D1 receptor agonist provides an improvement in treatment of depression; and a kit for screening for a dopamine D1 receptor agonist that in combination with an antidepressant provides an improvement in treatment of depression.

### Solution to Problem

The present inventors found out that chronic administration of an antidepressant alters the gene expression and neuronal functions in mature granule cells of mouse hippocampal dentate gyrus (mature dentate gyrus) to those like the ones in immature granule cells and that the expression of a dopamine D1 receptor significantly increases in the granule cells of the dematured dentate gyrus. Further, the inventors found out that, as a result of functional analysis, an antidepressant increases the dopamine D1 receptor signaling in the dentate gyrus; that combined administration of a dopamine D1 receptor agonist and an antidepressant (chronic administration) results in a significant increase in gene expression in the dentate gyrus; and that many of such genes exhibiting a significant increase in expression level are related to depression and the expression thereof is decreased due to depression (hereinafter sometimes referred to as “depression-related genes”). The inventors thus completed the present invention. Meanwhile, excessive activation of the dopamine D1 receptor may cause excessive excitation of granule cells, resulting in induction of seizure or manic state as a side effect of an antidepressant.

That is, the present invention relates to:

[1] a combination medicine for treatment of depression, comprising a combination of (A1) an antidepressant and either (B1) a dopamine D1 receptor agonist or (C1) a dopamine D1 receptor antagonist,
[2] the combination medicine according to the above [1], wherein combined use of (A1) the antidepressant and (B1) the dopamine D1 receptor agonist enhances an increase in dopamine D1 receptor signaling and in depression-related gene expression,
[3] the combination medicine according to the above [1] or [2], wherein (A1) the antidepressant is a sustained-release preparation,
[4] the combination medicine according to any one of the above [1] to [3], wherein (A1) the antidepressant is (a1) a tricyclic antidepressant, (a2) a tetracyclic antidepressant, (a3) a selective serotonin reuptake inhibitor, or (a4) a selective serotonin and/or noradrenaline reuptake inhibitor,
[5] the combination medicine according to the above [4], wherein the selective serotonin reuptake inhibitor is fluoxetine, fluvoxamine, sertraline, paroxetine or escitalopram,
[6] the combination medicine according to the above [1], wherein (B1) the dopamine D1 receptor agonist is SKF81297, SKF83959 or SKF38393,
[7] a pharmaceutical composition, comprising a combination of (A1) an antidepressant and either (B1) a dopamine D1 receptor agonist or (C1) the dopamine D1 receptor antagonist,
[8] a method for treatment of depression, comprising the steps of: administering a therapeutically effective amount of (A1) an antidepressant to a mammal, and administering a therapeutically effective amount of (B1) a dopamine D1 receptor agonist or (C1) a dopamine D1 receptor antagonist to the mammal simultaneously with or after the start of the administration of the antidepressant,
[9] a method for administering an antidepressant and either a dopamine D1 receptor agonist or a dopamine D1 receptor antagonist for treatment of depression, the method comprising the steps of: administering a therapeutically effective amount of (A1) an antidepressant to a mammal, and administering a therapeutically effective amount of either (B1) a dopamine D1 receptor agonist or (C1) a dopamine D1 receptor antagonist to the mammal simultaneously with or after the start of the administration of the antidepressant,
[10] the method according to the above [9], wherein the antidepressant is administered in the form of a sustained-release preparation,
[11] use of (A1) an antidepressant and either (B1) a dopamine D1 receptor agonist or (C1) a dopamine D1 receptor antagonist for the manufacture of medicine for treatment of depression,
[12] a method for screening for an antidepressant that in combination with a dopamine D1 receptor agonist provides an improvement in treatment of depression, the method comprising the steps of: administering, to a mammal, (A2) a compound having an antidepressant action and (B1) a dopamine D1 receptor agonist, and detecting a greater increase in depression-related gene expression in comparison with the case where (A2) the compound having an antidepressant action or (B1) the dopamine D1 receptor agonist is solely administered,
[13] the method according to the above [12]; wherein the administration of (A2) the compound having an antidepressant action and (B1) the dopamine D1 receptor agonist shows an increase in depression-related gene expression greater than the increase shown by sole administration of (A2) the compound having an antidepressant action and greater than the increase shown by sole administration of (B1) the dopamine D1 receptor agonist,
[14] the method according to the above [12], wherein the depression-related gene is any one or more selected from the group consisting of dopamine D1 receptor, p11, Annexin A2, tissue plasminogen activator, ARC, neuropeptide Y and BDNF,
[15] the method according to the above [12], wherein the depression-related gene expression is detected based on mRNA or protein expression of the depression-related gene,
[16] a method for screening for a dopamine D1 receptor agonist that in combination with an antidepressant provides an improvement in treatment of depression, the method comprising the steps of: administering, to a mammal, (A1) an antidepressant and (B2) a compound having a dopamine D1 receptor activating action, and detecting a greater increase in depression-related gene expression in comparison with the case where (A1) the antidepressant or (B2) the compound having a dopamine D1 receptor activating action is solely administered,
[17] a method for screening for an antidepressant that in combination with a dopamine D1 receptor agonist provides an improvement in treatment of depression, the method comprising the steps of: administering, to a mammal, (A2) a compound having an antidepressant action and (B1) a dopamine D1 receptor agonist, and detecting a greater increase in dopamine D1 receptor expression and/or dopamine D1 receptor signaling in comparison with the case where (A2) the compound having an antidepressant action or (B1) the dopamine D1 receptor agonist is administered,
[18] the method according to the above [17], wherein the administration of (A2) the compound having an antidepressant action and (B1) the dopamine D1 receptor agonist shows a greater increase in dopamine D1 receptor expression and/or dopamine D1 receptor signaling than the increase shown by the administration of (A2) the compound having an antidepressant action or (B1) the dopamine D1 receptor agonist,
[19] the method according to the above [17], wherein the dopamine D1 receptor expression is detected based on mRNA or protein expression of dopamine D1 receptor,
[20] the method according to the above [17], wherein the dopamine D1 receptor signaling is detected based on phosphorylation of DARPP-32 or ERK,
[21] a method for screening for a dopamine D1 receptor agonist that in combination with an antidepressant provides an improvement in treatment of depression, the method comprising the steps of: administering, to a mammal, (A1) an antidepressant and (B2) a compound having a dopamine D1 receptor activating action, and detecting a greater increase in dopamine D1 receptor expression and/or dopamine D1 receptor signaling in comparison with the case where (A1) the antidepressant or (B2) the compound having a dopamine D1 receptor activating action is administered,
[22] a kit for screening for an antidepressant that in combination with either a dopamine D1 receptor agonist or a dopamine D1 receptor antagonist provides an improvement in treatment of depression, the kit comprising either (B1) a, dopamine D1 receptor agonist or (C1) a dopamine D1 receptor antagonist, and either or both of (D1) a detection reagent for a depression-related gene and (E1) a detection reagent for dopamine D1 receptor signaling, and
[23] a kit for screening for a dopamine D1 receptor agonist that in combination with an antidepressant provides an improvement in treatment of depression, the kit comprising (A1) an antidepressant, and either or both of (D1) a detection reagent for a depression-related gene and (E1) a detection reagent for dopamine D1 receptor signaling.

### Advantageous Effects of Invention

According to the present invention, provided is a combination medicine for treatment of depression, using (A1) an antidepressant and either (B1) a dopamine D1 receptor agonist or (C1) a dopamine D1 receptor antagonist. Also, according to the present invention, provided is a pharmaceutical composition comprising a combination of (A1) an antidepressant and either (B1) a dopamine D1 receptor agonist or (C1) a dopamine D1 receptor antagonist. Further, according to the present invention, provided is a method for administration for treatment of depression, comprising the steps of: administering a therapeutically effective amount of an antidepressant to a mammal, and administering a therapeutically effective amount of (B1) a dopamine D1 receptor agonist or (C1) a dopamine D1 receptor antagonist to the mammal simultaneously with or after the start of the administration of the antidepressant. Also, according to the present invention, provided is use of (A1) an antidepressant and either (B1) a dopamine D1 receptor agonist or (C1) a dopamine D1 receptor antagonist for the manufacture of medicine for treatment of depression. Further, according to the present invention, provided is a method for screening for an antidepressant that in combination with a dopamine D1 receptor agonist provides an improvement in treatment of depression. Also, according to the present invention, provided is a method for screening for a dopamine D1 receptor agonist that in combination with an antidepressant provides an improvement in treatment of depression. Further, according to the present invention, provided is a kit for screening for an antidepressant that in combination with a dopamine D1 receptor agonist or antagonist provides an improvement in treatment of depression, the kit comprising either (B1) a dopamine D1 receptor agonist or (C1) a dopamine D1 receptor antagonist and either or both of (D1) a detection reagent for a depression-related gene and (E1) a detection reagent for dopamine D1 receptor signaling. Further, according to the present invention, provided is a kit for screening for a dopamine D1 receptor agonist that in combination with an antidepressant provides an improvement in treatment of depression, the kit comprising (A1) an antidepressant and either or both of (D1) a detection reagent for a depression-related gene and (E1) a detection reagent for dopamine D1 receptor signaling.

Also, combined use of (A1) an antidepressant with either (B1) a dopamine D1 receptor agonist or (C1) a dopamine D1 receptor antagonist enhances the action of the antidepressant and improves the problems of the antidepressant. Specifically, effects, such as rapid action of the antidepressant, improvement in the improvement rate and the cure rate of depression, improvement in the symptoms of depression in refractory depression, and inhibition of recurrence of depression, are exerted.

### BRIEF DESCRIPTION OF DRAWINGS

FIG. 1 shows mRNA expression in the hippocampal dentate gyrus of mice which were treated for 14 days with a subcutaneously implanted fluoxetine pellet or a placebo pellet as a control. The mRNA expression was analyzed by RT-PCR. As compared to the control, in the cases where the mice were treated with a fluoxetine pellet, the mRNA expression of dopamine D1 receptor increased, but the mRNA expression of calbindin, desmoplakin, tryptophan 2,3-dioxygenase, and interleukin-1 receptor decreased. These mRNA expression patterns are similar to those of immature hippocampus dentate gyrus, and thus dematured dentate gyrus induced by fluoxetine was confirmed. In FIG. 1, A.U. means arbitrary unit, and the horizontal axis of each chart shows treatment (*P<0.05, **P<0.01; P is a value in the case where a significant difference is shown by a T-test).

FIG. 2 shows analysis results of the dopamine D1 receptor signaling in the dematured dentate gyrus induced by fluoxetine. On slices of the dentate gyrus of mice which were treated for 14 days with a subcutaneously implanted fluoxetine pellet or a placebo pellet, a dopamine D1 receptor agonist, namely SKF81297 (1 uM or 10 uM), was made to act, and phosphorylation of DARPP-32 Thr34 was analyzed. SKF81297-induced phosphorylation of DARPP-32 was increased in the mice treated by chronic fluoxetine administration. In this figure, control means non-treated slices, control-P means placebo pellet, and fluoxetine-P means fluoxetine pellet. The left chart shows Thr34-phosphorylated DARPP-32, the central chart shows Thr34-phosphorylated DARPP-32 after adjustment with the amount of DARPP-32 protein, and the right chart shows the amount of DARPP-32 protein (*P<0.05, **P<0.01; P is a value in the case where a significant difference is shown by a T-test).

FIG. 3 shows analysis results of the dopamine D1 receptor expression in the dematured dentate gyrus induced by fluoxetine (analyzed using dopamine D1 receptor-GFP mice expressing GFP as a result of activation of dopamine D1 receptor promoter). Dopamine D1 receptor-GFP mice were treated for 14 days with a subcutaneously implanted fluoxetine pellet or a placebo pellet, and cells expressing GFP were analyzed (The upper row shows the expression of each protein in the hippocampal dentate gyrus of a mouse treated with a placebo pellet, and the lower row shows the expression of each protein in the hippocampal dentate gyrus of a mouse treated with a fluoxetine pellet. From the left, are shown nuclei, the expression of GFP induced by activation of dopamine D1 receptor promoter, the expression of calbindin (−D28K) as a mature granular cell marker, and a merged image of the three, and X40 means the object lens magnification. In mature granule cells expressing calbindin, the expression of GFP induced by activation of dopamine D1 receptor promoter is very low. However, in granule cells in the dentate gyrus dematured by chronic fluoxetine administration, the expression of calbindin is lowered and the expression of GFP induced by activation of dopamine D1 receptor promoter is increased.

FIG. 4 shows the results of microarray analysis and clustering analysis presenting gene expression enhancing action of dopamine D1 receptor agonist in the dematured dentate gyrus induced by fluoxetine (In this figure, are shown microarray analysis and clustering analysis on administration of control (C), dopamine D1 receptor agonist (SKF), fluoxetine (Fluox), and fluoxetine together with dopamine D1 receptor agonist (Fluox+SKF); Shown on the right are gene names used in the microarray analysis and clustering analysis; Red means gene expression higher than average, green means gene expression lower than average, and black means gene expression of an average level). Administration of a dopamine D1 receptor agonist (SKF81297; 3 mg/(kg day), i.p. for 5 days) to mice treated by chronic fluoxetine administration significantly enhanced gene expression in the dematured dentate gyrus as compared to control, administration of the dopamine D1 receptor agonist alone, and administration of fluoxetine alone.

FIG. 5 shows a model of dopamine D1 receptor function promotion in the dematured dentate gyrus induced by fluoxetine. In this figure, SGZ means the hippocampal dentate gyrus. In the dematured dentate gyrus induced by fluoxetine, the expression of the dopamine D1 receptor in granule cells is increased, and, in cooperation with fluoxetine, activation of the dopamine D1 receptor by a dopamine D1 receptor agonist significantly promotes the gene expression in the dentate gyrus. The results suggest that the function of the dopamine D1 receptor plays an important role for the expression of the action of the antidepressant.

FIG. 6 shows mRNA expression in the hippocampal dentate gyrus of mice which were treated for 14 days with a subcutaneously implanted imipramine pellet or a placebo pellet as a control. The mRNA expression was analyzed by RT-PCR. As compared to the control, in the cases where the mice were treated with a imipramine pellet, the mRNA expression of dopamine D1 receptor and BDNF increased, but the mRNA expression of calbindin, desmoplakin, tryptophan 2,3-dioxygenase, and interleukin-1 receptor decreased. From these results, as in the case of fluoxetine administration, dematured dentate gyrus induced by imipramine was confirmed. In FIG. 6, A.U. means arbitrary unit, and the horizontal axis of each chart shows treatment (*P<0.05, **P<0.01; P is a value in the case where a significant difference is shown by a T-test).

FIG. 7 shows mRNA expression in the hippocampal dentate gyrus of chronic fluoxetine administration model mice treated by administration of a dopamine D1 receptor agonist, namely SKF81297 (combination use). The level of mRNA expression was analyzed by RT-PCR. Combination use of fluoxetine and SKF81297 increased the mRNA expression levels of dopamine D1 receptor, BDNF, ARC, and neuropeptide Y (NPY), as compared to control, administration of SKF81297 alone, and administration of fluoxetine alone. The expression level of calbindin was inhibited as compared to control, administration of SKF81297 alone, and administration of fluoxetine alone. Therefore, it was revealed that combination use of SKF81297 further increases the depression-related gene expression increased by fluoxetine. In this figure, D2R means dopamine D2 receptor, SKF means SKF81297, FLX means fluoxetine, and SAL means physiological saline. In FIG. 7, A.U. means arbitrary unit, and the horizontal axis of each chart shows treatment (*P<0.05, ***P<0.001; P is a value in the case where a significant difference is shown by a multiple comparison test following a one-way analysis of variance).

FIG. 8 shows protein expression in the hippocampal dentate gyrus of chronic fluoxetine administration model mice treated by administration of a dopamine D1 receptor agonist, namely SKF81297 (combination use). The protein expression was analyzed by western blotting. Combination use of fluoxetine and SKF81297 increased the protein expression levels of dopamine D1 receptor, mBDNF, proBDNF, p11, and annexin A2, as compared to control, administration of SKF81297 alone, and administration of fluoxetine alone. In this figure, AxA2 means annexin A2, SKF means SKF81297, FLX means fluoxetine, and SAL means physiological saline. In FIG. 8, A.U. means arbitrary unit, and the horizontal axis of each chart shows treatment (*P<0.05, **P<0.01, ***P<0.001; P is a value in the case where a significant difference is shown by a multiple comparison test following a one-way analysis of variance).

### DESCRIPTION OF EMBODIMENTS

The present invention is based on the inventors' findings that a certain antidepressant increases the expression of dopamine D1 receptor and a certain dopamine D1 receptor agonist activates the dopamine D1 receptor, resulting in a greater increase in the expression of depression-related genes in the hippocampal dentate gyrus. Also, since such greater increase in the expression of a depression-related gene in the hippocampal dentate gyrus was not observed in the case of administration of the antidepressant alone or the dopamine D1 receptor agonist alone, the greater increase in the expression of a depression-related gene in the hippocampal dentate gyrus seems to be due to synergetic effect resulting from combined administration of the antidepressant and the dopamine D1 receptor agonist.

In the present invention, depression includes unipolar depression, bipolar depression (also referred to as bipolar disorder or manic depression), refractory depression, and the like.

The present invention relates to a combination medicine for treatment of depression, comprising a combination of (A1) an antidepressant and either (B1) a dopamine D1 receptor agonist or (C1) a dopamine D1 receptor antagonist. In the present invention, “combination medicine” means (1) a single preparation which is prepared at a time from (A1) an antidepressant and either (B1) a dopamine D1 receptor agonist or (C1) a dopamine D1 receptor antagonist, and (2) two or more kinds of preparations which are separately prepared from (A1) an antidepressant and either (B1) a dopamine D1 receptor agonist or (C1) a dopamine D1 receptor antagonist. The present invention is characterized in that combined use of (A1) an antidepressant and (B1) a dopamine D1 receptor agonist enhances an increase in dopamine D1 receptor signaling and in depression-related gene expression. In the case of hypomanic phase repetition (rapid cycling) caused by an antidepressant in bipolar depression, in the case of side effects, such as seizure, confusion, hallucination, and delirium caused by an antidepressant, or in order to prevent side effects of antidepressants or hypomanic phase repetition caused by an antidepressant, preferred is a combination medicine for treatment of depression, comprising a combination of (A1) an antidepressant and (C1) a dopamine D1 receptor antagonist. The combination medicine of the present invention also include a preparation comprising (A2) a compound having an antidepressant action instead of (A1) the antidepressant, a preparation comprising (B2) a compound having a dopamine D1 receptor activating action instead of (B1) the dopamine D1 receptor agonist, and a preparation comprising (C2) a compound having a dopamine D1 receptor antagonistic action instead of (C1) the dopamine D1 receptor antagonist. The (C2) compound having a dopamine D1 receptor antagonistic action may be, for example, the dopamine D1 receptor antagonist shown below, a novel compound having a dopamine D1 receptor antagonistic action, or the like.

Examples of the combination of ingredients in the combination medicine of the present invention include (A1) and (B1), (A1) and (C1), (A2) and (B1), (A2) and (C1), (A1) and (B2), (A1) and (C2), (A2) and (B2), and (A2) and (C2).

In the combination medicine of the present invention, (A1) the antidepressant is preferably (a1) a tricyclic antidepressant, (a2) a tetracyclic antidepressant, (a3) a selective serotonin reuptake inhibitor, or (a4) a selective serotonin and/or noradrenaline reuptake inhibitor; more preferably (a3) a selective serotonin reuptake inhibitor or (a4) a selective serotonin and/or noradrenaline reuptake inhibitor; and still more preferably (a3) a selective serotonin reuptake inhibitor.

In the combination medicine of the present invention, the tricyclic antidepressant may be any one as long as it inhibits reuptake of noradrenalin and serotonin. Examples of the tricyclic antidepressant include imipramine (product name: Imidol (registered trademark), product name: Tofranil (registered trademark)), amitriptyline (product name: Tryptanol (registered trademark), product name: Lantron (registered trademark)), and the like, but are not limited thereto.

In the combination medicine of the present invention, the tetracyclic antidepressant may be any one as long as it selectively inhibits reuptake of noradrenalin but does not inhibit reuptake of serotonin. Examples of the tetracyclic antidepressant include maprotiline (product name: Ludiomil (registered trademark)), mianserin (product name: Tetramide (registered trademark)), setiptiline (product name: Tecipul (registered trademark)), and the like, but are not limited thereto.

In the combination medicine of the present invention, the selective serotonin reuptake inhibitor may be any one as long as it selectively inhibits reuptake of serotonin and at higher doses inhibits reuptake of noradrenalin. Examples of the selective serotonin reuptake inhibitor include fluoxetine (product name: Prozac (registered trademark)), fluvoxamine (product name: Depromel (registered trademark), product name: Luvox (registered trademark)), sertraline (product name: JZoloft (registered trademark)), paroxetine (product name: Paxil (registered trademark)), escitalopram (product name: Lexapro (registered trademark), Cipralex (registered trademark)), and the like. Inter alia, the selective serotonin reuptake inhibitor is preferably fluoxetine (product name: Prozac (registered trademark)).

In the combination medicine of the present invention, the selective serotonin and/or noradrenaline reuptake inhibitor may be any one as long as it selectively inhibits reuptake of serotonin and at higher doses inhibits reuptake of noradrenalin. Examples of the selective serotonin and/or noradrenaline reuptake inhibitor include milnacipran (product name: Toledomin (registered trademark)), duloxetine (product name: Cymbalta (registered trademark)), and the like, but are not limited thereto.

In the combination medicine of the present invention, the above-mentioned antidepressant is preferably a sustained-release preparation. The sustained-release preparation achieves, for example, chronic administration of an antidepressant, and is suitable for inducing dematuration of the hippocampal dentate gyrus. The sustained-release preparation may be, for example, pellets custom-made by Innovative Research of America, or the like. The technology relating to sustained-release preparations has already been fully established, and the sustained-release preparation used in the present invention may be one manufactured according to such conventional technology.

In the combination medicine of the present invention, (B1) the dopamine D1 receptor agonist is not particularly limited as long as the agonist activates a dopamine D1 receptor. The dopamine D1 receptor agonist in the present invention is used as a selective dopamine D1 receptor agonist. Examples of the dopamine D1 receptor agonist include SKF81297 (Sigma, #S179; R-(+)-6-chloro-7,8-dihydroxy-1-phenyl-2,3,4,5-tetrahydro-1H-3-benzazepine hydrobromide), SKF83959 (Sigma, #S2816; 6-Chloro-7,8-dihydroxy-3-methyl-1-(3-methylphenyl)-2,3,4,5-tetrahydro-1H-3-benzazepine hydrobromide), SKF38393 (Sigma, #S101; (R)-(+)-SKF-38393 hydrochloride; another name: [R,(+)]-2,3,4,5-tetrahydro-1beta-phenyl-1H-3-benzazepine-7,8-diol hydrochloride), and the like. Inter alia, the dopamine D1 receptor agonist is preferably SKF81297, which shows a better effect of the present invention. In the present invention, the dopamine D1 receptor may be stated as D1R or D1 receptor. Examples of medicines that show actions similar to those of dopamine D1 receptor agonists include antiparkinson drugs, such as L-DOPA and pergolide; methylphenidate, which has been excluded from approved antidepressants since it causes dependence; a selective dopamine reuptake inhibitor; serotonin; norepinephrine; a dopamine reuptake inhibitor; and the like.

In the combination medicine of the present invention, (C1) the dopamine D1 receptor antagonist is not particularly limited as long as the antagonist has a dopamine D1 receptor antagonistic action. Examples of the dopamine D1 receptor antagonist include SCH-23390 (Sigma, #D054; R(+)-7-chloro-8-hydroxy-3-methyl-1-phenyl-2,3,4,5-tetrahydro-1H-3-benzazepine hydrochloride), SCH-12679 (Sigma, #S159; R(−)-1-phenyl-2,3,4,5-tetrahydro-1H-7,8-dimethoxy-3-benzazepine), LE300 (Sigma, #L8401; 7-methyl-6,7,8,9,14,15-hexahydro-5H-benz[d]indolo[2,3-g]azecine), and the like. Inter alia, for a better effect of the present invention, the dopamine D1 receptor antagonist is preferably SCH23390.

In the combination medicine of the present invention, the amount of the antidepressant (for example, fluoxetine) can be suitably selected depending on the targeted subject, the administration method, the combination of medicines, or the like, but the amount in the preparation is preferably 0.1 mg/kg to 250 mg/kg, more preferably 1 mg/kg to 60 mg/kg, still more preferably 5 mg/kg to 30 mg/kg per day. Also, the preparation may be one which can be administered in such a manner that the amount of the antidepressant can be gradually increased depending on the symptoms and the severity of the subject.

Regarding the combination medicine of the present invention, the antidepressant may be administered until the action thereof is exerted, and the above amount is administered for preferably 1 to 180 days, more preferably 3 to 60 days, and still more preferably 7 to 21 days. Also, the antidepressant in the above amount may be administered every day for the above period of time, or until the expression of the dopamine D1 receptor is more greatly increased due to the action of the antidepressant. In the present invention, the administration of the antidepressant continued until the action thereof is exerted, morphological change of dematuration of the hippocampal dentate gyrus is shown, or the expression of the dopamine D1 receptor is increased may be referred to as “chronic administration.”

In the combination medicine of the present invention, the amount of the dopamine D1 receptor agonist (for example, SKF81297) can be suitably selected depending on the targeted subject, the administration method, the combination of medicines, or the like, but the amount in the preparation is preferably 0.05 mg/kg to 50 mg/kg, more preferably 0.1 mg/kg to 20 mg/kg, still more preferably 0.3 mg/kg to 10 mg/kg per day. Also, the preparation may be one which can be administered in such a manner that the amount of the antidepressant can be gradually increased depending on the symptoms and the severity of the subject. The amount of the dopamine D1 receptor antagonist is also the same as that of the dopamine D1 receptor agonist.

Regarding the combination medicine of the present invention, the dopamine D1 receptor agonist or the dopamine D1 receptor antagonist is administered for preferably 1 to 180 days, more preferably 1 to 60 days, and still more preferably 1 to 21 days. Also, the dopamine D1 receptor agonist or the dopamine D1 receptor antagonist in the above amount may be administered every day for the above period of time.

In the combination medicine of the present invention, (A1) the antidepressant and either (B1) the dopamine D1 receptor agonist or (C1) the dopamine D1 receptor antagonist may be formed into a mixed preparation, but may also be separately formulated. In the combination medicine, the contents of the antidepressant and the dopamine D1 receptor agonist may be the same as described above, and the administration period may also be as described above.

In the combination medicine of the present invention, the ratio of (A1) the antidepressant to either (B1) the dopamine D1 receptor agonist or (C1) the dopamine D1 receptor antagonist (the amount of (A1) the antidepressant used/the amount of either (B1) the dopamine D1 receptor agonist or (C1) the dopamine D1 receptor antagonist used) is preferably 1/0.001 to 1/100, more preferably 1/0.01 to 1/10, and still more preferably 1/0.05 to 1/1, and these ratios may be per day.

The combination medicine of the present invention can be mixed with a pharmaceutically acceptable carrier and formed into a tablet, a granule, a capsule, a liquid, a suppository, a sustained-release preparation, a pellet, or the like, according to a production method publicly known in the pharmaceutical field. Examples of the pharmaceutically acceptable carrier include, for example, an organic carrier, an inorganic carrier, a lubricant, a binder, a disintegrator, an excipient, a suspending agent, a tonicity agent, and a buffer. The proportion of the carrier in the combination medicine can be suitably selected depending on the targeted subject, the administration route, or the like. The pharmaceutical technology has already been fully established, and such various known additives and pharmaceutical technology may be used in the present invention.

The administration method of the combination medicine of the present invention is, for example, (1) administration of a single preparation which is prepared at a time from (A1) an antidepressant and either (B1) a dopamine D1 receptor agonist or (C1) a dopamine D1 receptor antagonist, (2) administration of two or more kinds of preparations which are separately prepared from (A1) an antidepressant and either (B1) a dopamine D1 receptor agonist or (C1) a dopamine D1 receptor antagonist using a single administration route, (3) administration of two or more kinds of preparations which are separately prepared from (A1) an antidepressant and either (B1) a dopamine D1 receptor agonist or (C1) a dopamine D1 receptor antagonist using a single administration route at different times, (4) administration of two or more kinds of preparations which are separately prepared from (A1) an antidepressant and either (B1) a dopamine D1 receptor agonist or (C1) a dopamine D1 receptor antagonist using different administration routes at a time, (5) administration of two or more kinds of preparations which are separately prepared from (A1) an antidepressant and either (B1) a dopamine D1 receptor agonist or (C1) a dopamine D1 receptor antagonist using different administration routes at different times, or the like. Regarding the combination medicine of the present invention, administration of the above (3) or (5) is preferred, and (5) is more preferred. In the above (5), the antidepressant may be administered before or after the administration of either (B1) the dopamine D1 receptor agonist or (C1) a dopamine D1 receptor antagonist.

In the case of (3) or (5), where administration is performed at different times, the time difference varies with the targeted subject, the ingredients to be administered, the formulation, or the like. For example, in the case where the antidepressant is administered first and then either (B1) the dopamine D1 receptor agonist or (C1) a dopamine D1 receptor antagonist is administered, the administration of either (B1) the dopamine D1 receptor agonist or (C1) a dopamine D1 receptor antagonist may be started preferably within 1 to 90 days, and more preferably 3 to 14 days from the administration of the antidepressant, and it is also allowable that the administration of the antidepressant is continued while either (B1) the dopamine D1 receptor agonist or (C1) a dopamine D1 receptor antagonist is administered.

Regarding the combination medicine of the present invention, examples of the “administration” include, for example, oral administration and parenteral administration. Examples of the parenteral administration include subcutaneous, intravenous, intraarterial, intranodular, intramedullary, intraspinal, intraventricular, intranasal, intrabronchial, transdermal, intrarectal, intraperitoneal, intramuscular, intrapulmonary, intravaginal, and intraocular administration, and the like. “Administration” also means embedding, implant, or the like of the combination medicine into the body, and may be referred to as “treatment.”

The present invention relates to a pharmaceutical composition, comprising a combination of (A1) an antidepressant and either (B1) a dopamine D1 receptor agonist or (C1) a dopamine D1 receptor antagonist. (A1) The antidepressant and either (B1) the dopamine D1 receptor agonist or (C1) the dopamine D1 receptor antagonist may be as described above, and the amounts used, the administration period, the administration method, or the like may be as described above. The pharmaceutical composition may be applied to other diseases than depression.

The present invention relates to a method for treatment of depression, comprising the steps of: administering a therapeutically effective amount of (A1) an antidepressant to a mammal, and administering a therapeutically effective amount of either (B1) a dopamine D1 receptor agonist or (C1) a dopamine D1 receptor antagonist to the mammal simultaneously with or after the start of the administration of the antidepressant. In the present invention, “therapeutically effective amount” means an amount of (A1) the antidepressant, (B1) the dopamine D1 receptor agonist, or (C1) the dopamine D1 receptor antagonist enough to show the effect of (A1) the antidepressant, (B1) the dopamine D1 receptor agonist, or (C1) the dopamine D1 receptor antagonist when administered to a mammal. The “mammal” includes a human, an ape, a gorilla, a horse, a cow, a sheep, a dog, a cat, a rabbit, a rat, a mouse, and the like, and also includes sliced tissue of the hippocampus of these animals, etc.

In the method of the present invention for treatment of depression, (A1) the antidepressant, (B1) the dopamine D1 receptor agonist, or (C1) the dopamine D1 receptor antagonist may be the same as that described as used in the above combination medicine, and may be administered as the above combination medicine. Also, the administration period of the therapeutically effective amount of (A1) the antidepressant, the therapeutically effective amount of (B1) the dopamine D1 receptor agonist, the therapeutically effective amount of (C1) the dopamine D1 receptor antagonist, (A1) the antidepressant, (B1) the dopamine D1 receptor agonist, or (C1) the dopamine D1 receptor antagonist may be the same as described above.

In the method of the present invention for treatment of depression, the antidepressant is preferably administered in the form of a sustained-release preparation. Such a sustained-release preparation achieves, for example, chronic administration of an antidepressant, and is suitable for inducing dematuration of the hippocampal dentate gyrus. The sustained-release preparation may be the same as described above.

In the method of the present invention for treatment of depression, “administration” means the same as described above. For example, the antidepressant may be subcutaneously implanted as a pelletized sustained-release preparation, and the dopamine D1 receptor agonist may be intraperitoneally administered as a preparation in any form.

The present invention relates to a method for administering an antidepressant and either a dopamine D1 receptor agonist or a dopamine D1 receptor antagonist for treatment of depression, the method comprising the steps of: administering a therapeutically effective amount of (A1) an antidepressant to a mammal, and administering a therapeutically effective amount of either (B1) a dopamine D1 receptor agonist or (C1) a dopamine D1 receptor antagonist to the mammal simultaneously with or after the start of the administration of the antidepressant.

In the method for administration of the present invention, the antidepressant is preferably administered in the form of a sustained-release preparation. Such a sustained-release preparation achieves, for example, chronic administration of an antidepressant, and is suitable for inducing dematuration of the hippocampal dentate gyrus. The sustained-release preparation may be the same as described above.

In the method for administration of the present invention, (A1) the antidepressant, (B1) the dopamine D1 receptor agonist, or (C1) the dopamine D1 receptor antagonist may be administered as the combination medicine described above, and (A1) the antidepressant, (B1) the dopamine D1 receptor agonist, (C1) the dopamine D1 receptor antagonist, the therapeutically effective amount of (A1) the antidepressant, the therapeutically effective amount of (B1) the dopamine D1 receptor agonist, and the administration period of the antidepressant or the dopamine D1 receptor agonist may be the same as described above.

The present invention relates to use of (A1) an antidepressant and either (B1) a dopamine D1 receptor agonist or (C1) a dopamine D1 receptor antagonist for the manufacture of medicine for treatment of depression. (A1) The antidepressant, (B1) the dopamine D1 receptor agonist, (C1) the dopamine D1 receptor antagonist, the amounts used thereof, the administration method thereof, and the like may be as described above, and the medicine may be a single preparation or two or more kinds of preparations depending on the targeted subject, the administration route, the combination of ingredients, or the like.

The present invention relates to a method for screening for an antidepressant that in combination with a dopamine D1 receptor agonist provides an improvement in treatment of depression, the method comprising the steps of: [1] administering, to a mammal, (A2) a compound haying an antidepressant action and (B1) a dopamine D1 receptor agonist, and [2] detecting a greater increase in depression-related gene expression in comparison with the case where (A2) the compound having an antidepressant action or (B1) the dopamine D1 receptor agonist is solely administered. In the screening method, wherein a known dopamine D1 receptor agonist, for example SKF81297, is used as a research tool, the screening for antidepressants is performed using, as an indicator, that the combined administration of the compound having an antidepressant action and the dopamine D1 receptor agonist shows a greater increase in depression-related gene expression than the increase shown by separate administration of the compound having an antidepressant action or the dopamine D1 receptor agonist. In the present invention, (A2) the compound having an antidepressant action is such a compound as to have an antidepressant action and increase the depression-related gene expression when used together with (B1) a dopamine D1 receptor agonist.

In the screening method of the present invention, the administration of (A2) the compound having an antidepressant action and of (B1) the dopamine D1 receptor agonist preferably shows an increase in depression-related gene expression greater than the increase shown by sole administration of the compound having an antidepressant action and greater than the dopamine D1 receptor agonist.

In the screening method of the present invention, the step of administering, to a mammal, (A2) a compound having an antidepressant action and (B1) a dopamine D1 receptor agonist (hereinafter may be referred to as Combined Administration Step 1) may be a step of administering a sufficient amount of (B1) a dopamine D1 receptor agonist to a mammal to which a sufficient amount of (A2) a compound having an antidepressant action has been administered beforehand. Also, as a comparative control, a step of administering a sufficient amount of (A2) the compound having an antidepressant action to a mammal, administering a sufficient amount of (B1) the dopamine D1 receptor agonist, or administering (C1) the dopamine D1 receptor antagonist to a mammal may be performed. Here, the “sufficient amount” means an amount of (A2) the compound having an antidepressant action and (B1) the dopamine D1 receptor agonist, the amount being suitable for the screening for antidepressants. Further, in the screening method, instead of the above Combined Administration Step 1, a step of applying (for example, spraying, inoculating, adding, etc.) (A2) a compound having an antidepressant action and (B1) a dopamine D1 receptor agonist to isolated tissue or cells of a mammal may be performed. Examples of the isolated tissue or cells of a mammal include sliced tissue of the hippocampus, sliced tissue of the hippocampal dentate gyrus, the hippocampal dentate gyrus, mature neurons in the hippocampal dentate gyrus, and the like. In the isolated tissue or cells of a mammal, tissue or cells produced by induction of differentiation from an ES (embryonic stem) cell and/or an iPS (induced pluripotent stem) cell are also included.

(A2) The compound having an antidepressant action may be, for example, an above-mentioned antidepressant, or a novel compound having an antidepressant action. The dopamine D1 receptor agonist may be as described above, but is not limited thereto. The sufficient amount, the administration period, the administration method, and the like of the compound having an antidepressant action, or the dopamine D1 receptor agonist may be any as long as they are suitable for the screening, and may be as described above.

The step of detecting a greater increase in depression-related gene expression in comparison with the case where (A2) the compound having an antidepressant action or (B1) the dopamine D1 receptor agonist is solely administered, is preferably a step of detecting an increase in depression-related gene expression in a mammal to which (A2) the compound having an antidepressant action and (B1) the dopamine D1 receptor agonist have been administered. Also, as a comparative control, a step of detecting an increase in depression-related gene expression in a mammal to which (A2) the compound having an antidepressant action or (B1) the dopamine D1 receptor agonist has been administered alone may be performed. Such a comparative control step may be performed at the same time as Combined Administration Step 1, or before or after Combined Administration Step 1. In the case where the comparative control step is performed before Combined Administration Step 1, the level of the depression-related gene expression in each control administration may be detected and determined beforehand.

In the screening method of the present invention, examples of the depression-related gene include a dopamine D1 acceptor, p11 (S100A10), annexin A2, a tissue plasminogen activator, ARC (activity-regulated cytoskeletal-associated protein), neuropeptide Y and BDNF (brain-derived neurotrophic factor), but are not limited thereto. These may be used alone or in a combination of two or more thereof. The expression of the above-mentioned gene is preferably increased in the screening method. The depression-related gene may include calbindin. In the screening method, the expression of calbindin may be decreased, and a step of detecting a decrease in depression-related gene expression in a mammal to which (A2) a compound having an antidepressant action and/or (B1) a dopamine D1 receptor agonist has been administered may be performed.

The depression-related gene expression is preferably detected based on the mRNA or protein expression of the depression-related gene. The mRNA or protein expression of the depression-related gene is detectable by publicly known methods, such as RT-PCR, in situ hybridization, immunohistochemistry, western blotting, and the like.

“A greater increase in depression-related gene expression” means the following situation: the levels of the depression-related gene expression of (1) mammals to which (A2) a compound having an antidepressant action has been administered, (2) mammals to which (B1) a dopamine D1 receptor agonist has been administered, and (3) mammals to which (A2) the compound having an antidepressant action and (B1) the dopamine D1 receptor agonist have been administered, are each averaged and statistically processed, and the comparison shows that the level of the depression-related gene expression of (3) is relatively higher than those of (1) and (2). In the comparison, data obtained from what is called microarray analysis, clustering analysis, or the like may be used.

A microarray is a device for analysis of a nucleic acid using hybridization between a nucleic acid to be detected and a nucleic acid probe having a complementary sequence to the objective nucleic acid.

The microarray should comprise a substrate and nucleic acid probes fixed onto the substrate, and can be manufactured by a known method in the field. The number of probe-fixed fields on the substrate and the arrangement thereof may be suitably modified if needed by the skilled person. Such a microarray is preferably used in a detection method using fluorescence.

The hybridization should be performed in appropriate conditions which allow hybrids to be well formed. The appropriate conditions vary with the type and structure of the target nucleic acid, the type of bases comprised in the target sequence, and the type of nucleic acid probes. As the washing solution used for washing the microarray after hybridization, a buffer solution of an ionic strength of 0.01 to 5 and of a pH 5 to 9 is preferably used. The washing solution preferably comprises a salt, a surfactant, and the like. The washing temperature is, for example, 10° C. to 70° C. The washing solution is made to pass through or be retained on the surface of the substrate with fixed probes or the fields with fixed probes. Alternatively, a microarray may be soaked in the washing solution. In this case, the washing solution is preferably contained in a container of which the temperature is controllable.

In order to detect hybrids formed in the hybridization step, a fluorescence detection method and an electrochemical detection method can be used. For example, in the case where a fluorescently-labeled substance is used in the fluorescence detection method, the primers used in the nucleic acid amplification step may be labeled with a fluorescently-active substance, such as FITC, Cy3, Cy5, rhodamine, or the like, second probes labeled with such a substance may be used, and two or more labeling substances may be used at a time. With a detecting device, a labeled sequence or a label in a second probe is detected. A suitable detecting device is used depending on the label to be used. For example, in the case where a fluorescent substance is used as a label, a fluorescence detector is used for detection.

Regarding the greater increase in depression-related gene expression, the level of the depression-related gene expression of (3) mammals to which (A2) the compound having an antidepressant action and (B1) the dopamine D1 receptor agonist have been administered, is higher than those of (1) mammals to which the compound having an antidepressant action has been administered and (2) mammals to which the dopamine D1 receptor agonist has been administered, preferably by 1.1 to 200 times, more preferably by 1.5 to 100 times, and still more preferably by 2 to 50 times. The candidate medicines found by this screening are used for an animal experiment, a clinical application, or the like, through which an appropriate administration amount, an appropriate administration period, or the like can be determined depending on the targeted subject or the administration route.

The present invention relates to a method for screening for a dopamine D1 receptor agonist that in combination with an antidepressant provides an improvement in treatment of depression, the method comprising the steps of: [1] administering, to a mammal, (A1) an antidepressant and (B2) a compound having a dopamine D1 receptor activating action, and [2] detecting a greater increase in depression-related gene expression in comparison with the case where (A1) the antidepressant or (B2) the compound having a dopamine D1 receptor activating action is solely administered. In the screening method, wherein a known antidepressant, for example fluoxetine, is used as a research tool, the screening for a dopamine D1 receptor agonist is performed using, as an indicator, that the combined administration of (A1) an antidepressant and (B2) a compound having the action of dopamine D1 receptor agonist shows a greater increase in depression-related gene expression than the increase shown by separate administration of (A1) the antidepressant or (B2) the compound having the action of dopamine D1 receptor agonist. In the present invention, (B2) the compound having the action of dopamine D1 receptor agonist is such a compound as to have the action of dopamine D1 receptor agonist and increase the depression-related gene expression when used together with an antidepressant.

In the screening method of the present invention for a dopamine D1 receptor agonist, the administration of (A1) an antidepressant and (B2) a compound having the action of dopamine D1 receptor agonist preferably shows an increase in depression-related gene expression greater than the increase shown by sole administration of (A1) the antidepressant and greater than the increase shown by sole administration of (B2) the compound having the action of dopamine D1 receptor agonist.

In the screening method of the present invention, the step of administering, to a mammal, (A1) an antidepressant and (B2) a compound having the action of dopamine D1 receptor agonist (hereinafter may be referred to as Combined Administration Step 2) may be a step of administering a sufficient amount of (B2) a compound having the action of dopamine D1 receptor agonist to a mammal to which a sufficient amount of (A1) an antidepressant has been administered beforehand. Also, as a comparative control, a step of administering a sufficient amount of (A1) the antidepressant to a mammal or administering a sufficient amount of (B2) the compound having the action of dopamine D1 receptor agonist to a mammal may be performed. Here, the “sufficient amount” means an amount of (A1) the antidepressant or (B2) the compound having the action of dopamine D1 receptor agonist, the amount being suitable for the screening for (B1) dopamine D1 receptor agonists. Further, in the screening method, instead of the above Combined Administration Step 2, a step of applying (for example, spraying, inoculating, adding, etc.) (A1) the antidepressant and (B2) a compound having the action of dopamine D1 receptor agonist to isolated tissue or cells of a mammal may be performed. Examples of the isolated tissue or cells of a mammal include sliced tissue of the hippocampus, sliced tissue of the hippocampal dentate gyrus, the hippocampal dentate gyrus, mature neurons in the hippocampal dentate gyrus, and the like. In the isolated tissue or cells of a mammal, tissue or cells produced by induction of differentiation from an ES (embryonic stem) cell and/or an iPS (induced pluripotent stem) cell are also included.

(A1) The antidepressant used for the screening method of the present invention may be as described above, but is not limited thereto. (B2) The compound having the action of dopamine D1 receptor agonist may be, for example, an above-mentioned dopamine D1 receptor agonist, or a novel compound having the action of dopamine D1 receptor agonist. The sufficient amount, the administration period, the administration method, and the like of the antidepressant or the compound having the action of dopamine D1 receptor agonist may be any as long as they are suitable for the screening, and may be as described above.

The step of detecting a greater increase in depression-related gene expression in comparison with the case where (A1) the antidepressant or (B2) the compound having the action of dopamine D1 receptor agonist is solely administered, is preferably a step of detecting an increase in depression-related gene expression in a mammal to which (A1) the antidepressant and (B2) the compound having the action of dopamine D1 receptor agonist have been administered. Also, as a comparative control, a step of detecting an increase in depression-related gene expression in a mammal to which (A1) the antidepressant or (B2) the compound having the action of dopamine D1 receptor agonist has been administered alone may be performed. Such a comparative control step may be performed at the same time as Combined Administration Step 2, or before or after Combined Administration Step 2. In the case where the comparative control step is performed before Combined Administration Step 2, the level of the depression-related gene expression in each control administration may be detected and determined beforehand.

In the screening method of the present invention, the depression-related gene may be as described above. The depression-related gene may include calbindin. In the screening method, the expression of calbindin may be decreased, and a step of detecting a decrease in depression-related gene expression in a mammal to which (A1) an antidepressant and/or (B2) a compound having the action of dopamine D1 receptor agonist has been administered may be performed.

The depression-related gene expression and the detecting method thereof may be as described above.

“A greater increase in depression-related gene expression” means the following situation: the levels of the depression-related gene expression of (1) mammals to which (A1) an antidepressant has been administered, (2) mammals to which (B2) a compound having the action of dopamine D1 receptor agonist has been administered, and (3) mammals to which (A1) the antidepressant and (B2) the compound having the action of dopamine D1 receptor agonist have been administered, are each averaged and statistically processed, and the comparison shows that the level of the depression-related gene expression of (3) is relatively higher than those of (1) and (2). In the comparison, data obtained from what is called microarray analysis, clustering analysis, or the like may be used. The microarray, clustering, etc. may be the same as described above.

Regarding the greater increase in depression-related gene expression, the level of the depression-related gene expression of (3) mammals to which (A1) the antidepressant and (B2) the compound having the action of dopamine D1 receptor agonist have been administered, is higher than those of (1) mammals to which (A1) the antidepressant has been administered and (2) mammals to which (B2) the compound having the action of dopamine D1 receptor agonist has been administered, preferably by 1.1 to 200 times, more preferably by 1.5 to 100 times, and still more preferably by 2 to 50 times. The candidate medicines found by this screening are used for an animal experiment, a clinical application, or the like, through which an appropriate administration amount, an appropriate administration period, or the like can be determined depending on the targeted subject or the administration route.

The present invention relates to a method for screening for an antidepressant that in combination with (B1) a dopamine D1 receptor agonist provides an improvement in treatment of depression, the method comprising the steps of: administering, to a mammal, (A2) a compound having an antidepressant action and (B1) a dopamine D1 receptor agonist, and detecting a greater increase in dopamine D1 receptor expression and/or dopamine D1 receptor signaling in comparison with the case where (A2) the compound having an antidepressant action or (B1) the dopamine D1 receptor agonist is administered. In the screening method, wherein a known dopamine D1 receptor agonist, for example SKF81297, is used as a research tool, the screening for antidepressants is performed using, as an indicator, that the administration of (A2) the compound having an antidepressant action and (B1) the dopamine D1 receptor agonist shows a greater increase in the expression of the dopamine D1 receptor and/or in the signaling of the dopamine D1 receptor than the increase shown by the administration of (A2) the compound having an antidepressant action or (B1) the dopamine D1 receptor agonist. In this embodiment, the compound having an antidepressant action is such a compound as to have an antidepressant action and show a greater increase in the expression of the dopamine D1 receptor and/or the signaling of the dopamine D1 receptor when used together with a dopamine D1 receptor agonist. The “increase” means the case where the degree of phosphorylation (for example, DARPP-32 Thr-34) resulting from the administration of a compound having an antidepressant action and the dopamine D1 receptor agonist is greater than the phosphorylation resulting from the administration of the compound having an antidepressant action or the dopamine D1 receptor agonist.

It is preferred that the administration of (A2) the compound having an antidepressant action and (B1) the dopamine D1 receptor agonist shows a greater increase in dopamine D1 receptor expression and/or dopamine D1 receptor signaling than the increase shown by the administration of (A2) the compound having an antidepressant action or (B1) the dopamine D1 receptor agonist.

In the screening method of the present invention, the step of administering, to a mammal, (A2) a compound having an antidepressant action and (B1) a dopamine D1 receptor agonist (hereinafter may be referred to as Combined Administration Step 3) may be a step of administering a sufficient amount of (B1) a dopamine D1 receptor agonist to a mammal to which a sufficient amount of (A2) a compound having an antidepressant action has been administered beforehand. Also, as a comparative control, a step of administering a sufficient amount of (A2) the compound having an antidepressant action or a sufficient amount of (B1) the dopamine D1 receptor agonist to a mammal may be performed. Here, the “sufficient amount” means an amount of (A2) the compound having an antidepressant action or (B1) the dopamine D1 receptor agonist, the amount being suitable for the screening for antidepressants. Further, in the screening method, instead of the above Combined Administration Step 3, a step of applying (for example, spraying, inoculating, adding, etc.) (A2) the compound having an antidepressant action and (B1) a dopamine D1 receptor agonist to isolated tissue or cells of a mammal may be performed. Examples of the isolated tissue or cells of a mammal include sliced tissue of the hippocampus, sliced tissue of the hippocampal dentate gyrus, the hippocampal dentate gyrus, mature neurons in the hippocampal dentate gyrus, and the like. In the isolated tissue or cells of a mammal, tissue or cells produced by induction of differentiation from an ES (embryonic stem) cell and/or an iPS (induced pluripotent stem) cell are also included.

(A2) The compound having an antidepressant action may be, for example, an above-mentioned antidepressant, or a novel compound having an antidepressant action. The dopamine D1 receptor agonist may be the same as the above, but is not limited thereto. The sufficient amount, the administration period, the administration method, and the like of the compound having an antidepressant action or the dopamine D1 receptor agonist may be any as long as they are suitable for the screening, and may be the same as the above. In the case of detecting dopamine D1 receptor signaling, it is also allowable that an appropriate amount of a dopamine D1 receptor agonist is administered to a slice of the hippocampus of a mammal to which a compound having an antidepressant action has been administered (added). In such a case, the amount of the dopamine D1 receptor agonist is preferably 0.001 uM to 50 uM and more preferably 0.01 uM to 20 uM.

The step of detecting a greater increase in dopamine D1 receptor expression and/or dopamine D1 receptor signaling in comparison with the case where (A2) the compound having an antidepressant action or (B1) the dopamine D1 receptor agonist is administered is preferably a step of detecting a greater increase in dopamine D1 receptor expression and/or dopamine D1 receptor signaling in a mammal to which (A2) a compound having an antidepressant action and (B1) a dopamine D1 receptor agonist have been administered. Also, as a comparative control, a step of detecting a greater increase in dopamine D1 receptor expression and/or dopamine D1 receptor signaling in a mammal to which (A2) the compound having an antidepressant action or (B1) the dopamine D1 receptor agonist has been administered may be performed. Such a comparative control step may be performed at the same time as Combined Administration Step 3, or before or after Combined Administration Step 3. In the case where the comparative control step is performed before Combined Administration Step 3, the level of the dopamine D1 receptor expression and/or of dopamine D1 receptor signaling may be detected and determined beforehand.

The dopamine D1 receptor expression is detected based on mRNA or protein expression of dopamine D1 receptor. The mRNA or protein expression of the dopamine D1 receptor is detectable by publicly known methods, such as RT-PCR, in situ hybridization, immunohistochemistry, western blotting, and the like.

The dopamine D1 receptor signaling is preferably detected based on phosphorylation of DARPP-32 or ERK. The phosphorylation of DARPP-32 is preferably phosphorylation in Thr34 (PKA site). Examples of the detection method of phosphorylation include immunoblotting with a phosphorylation state-specific antibody; autoradiography by use of cells metabolically labeled with [³²P] orthophosphate; and the like.

“A greater increase in dopamine D1 receptor expression and/or dopamine D1 receptor signaling” means the following situation: the levels of the dopamine D1 receptor expression and/or dopamine D1 receptor signaling of (1) mammals to which (A2) the compound having an antidepressant action or (B1) a dopamine D1 receptor agonist has been administered, and (2) mammals to which (A2) the compound having an antidepressant action and (B1) the dopamine D1 receptor agonist have been administered, are each averaged and statistically processed, and the comparison shows that the level of the dopamine D1 receptor expression and/or dopamine D1 receptor signaling of (2) is relatively higher than that of (1).

Regarding the greater increase in dopamine D1 receptor expression and/or dopamine D1 receptor signaling, the level of the dopamine D1 receptor expression and/or dopamine D1 receptor signaling of (2) mammals to which (A2) the compound having an antidepressant action and (B1) the dopamine D1 receptor agonist have been administered, is higher than those of (1) mammals to which (A2) the compound having an antidepressant action or (B1) the dopamine D1 receptor agonist has been administered, preferably by 1.1 to 100 times, more preferably by 1.5 to 20 times, and still more preferably by 2.0 to 10 times. The candidate medicines found by this screening are used for an animal experiment, a clinical application, or the like, through which an appropriate administration amount, an appropriate administration period, or the like can be determined depending on the targeted subject or the administration route.

The screening method of this embodiment may be performed in the same way except that, instead of (A2) the compound having an antidepressant action, various compounds which increase the dopamine D1 receptor expression and/or dopamine D1 receptor signaling (for example, an atypical antipsychotic, a novel serotonin receptor agonist and antagonist, an anticonvulsant, a glucocorticoid receptor antagonist, a corticotropin release factor antagonist, etc.) and control agents for the various compounds are used. By this screening, various compounds can also be screened.

The present invention relates to a method for screening for a dopamine D1 receptor agonist that in combination with an antidepressant provides an improvement in treatment of depression, the method comprising the steps of: administering, to a mammal, (A1) an antidepressant and (B2) a compound having a dopamine D1 receptor activating action, and detecting a greater increase in dopamine D1 receptor expression and/or dopamine D1 receptor signaling in comparison with the case where (A2) the compound having an antidepressant action or (B2) the compound having a dopamine D1 receptor activating action is administered. In the screening method, wherein a known antidepressant, for example fluoxetine, is used as a research tool, the screening for dopamine D1 receptor agonist is performed using, as an indicator, that the administration of (A1) an antidepressant and (B2) a compound having the action of dopamine D1 receptor agonist shows a greater increase in the expression of the dopamine D1 receptor and/or the signaling of the dopamine D1 receptor than the increase shown by administration of the antidepressant or (B2) the compound having the action of dopamine D1 receptor agonist. In this embodiment, (B2) the compound having the action of dopamine D1 receptor agonist is such a compound as to have the action of dopamine D1 receptor agonist and show a greater increase in the expression of the dopamine D1 receptor and/or the signaling of the dopamine D1 receptor when used together with an antidepressant. The “increase” means the case where the degree of phosphorylation (for example, DARPP-32 Thr-34) resulting from the administration of an antidepressant and the dopamine D1 receptor agonist is greater than the phosphorylation resulting from the administration of (A1) the antidepressant or the dopamine D1 receptor agonist.

In the screening method of the present invention for a dopamine D1 receptor agonist, it is preferred that the administration of (A1) the antidepressant and (B2) the compound having the action of dopamine D1 receptor agonist shows a greater increase in dopamine D1 receptor expression and/or dopamine D1 receptor signaling than the increase shown by the administration of (A1) the antidepressant or the compound having the action of dopamine D1 receptor agonist.

In the screening method of the present invention, the step of administering, to a mammal, (A1) an antidepressant and (B2) a compound having the action of dopamine D1 receptor agonist (hereinafter may be referred to as Combined Administration Step 4) may be a step of administering a sufficient amount of (B2) the compound having the action of dopamine D1 receptor agonist to a mammal to which a sufficient amount of (A1) the antidepressant has been administered beforehand. Also, as a comparative control, a step of administering a sufficient amount of (A1) the antidepressant or a sufficient amount of (B2) the compound having the action of dopamine D1 receptor agonist to a mammal may be performed. Here, the sufficient amount means an amount of (A1) the antidepressant or (82) the compound having the action of dopamine D1 receptor agonist, the amount being suitable for the screening for dopamine D1 receptor agonists or dopamine D1 receptor antagonists. Further, in the screening method, instead of the above Combined Administration Step 4, a step of applying (for example, spraying, inoculating, adding, etc.) (A1) the antidepressant and (B2) a compound having the action of dopamine D1 receptor agonist to isolated tissue or cells of a mammal may be performed. Examples of the isolated tissue or cells of a mammal include sliced tissue of the hippocampus, sliced tissue of the hippocampal dentate gyrus, the hippocampal dentate gyrus, mature neurons in the hippocampal dentate gyrus, and the like. In the isolated tissue or cells of a mammal, tissue or cells produced by induction of differentiation from an ES (embryonic stem) cell and/or an iPS (induced pluripotent stem) cell are also included.

(A1) The antidepressant used for the screening method may be, for example, the same as the above, but is not limited thereto. (B2) The compound having the action of dopamine D1 receptor agonist may be, for example, an above-mentioned dopamine D1 receptor agonist, or a novel compound having the action of dopamine D1 receptor agonist. The sufficient amount, the administration period, the administration method, and the like of (A1) the antidepressant or (B2) the compound having the action of dopamine D1 receptor agonist may be any as long as they are suitable for the screening, and may be the same as the above. In the case of detecting dopamine D1 receptor signaling, it is also allowable that an appropriate amount of a dopamine D1 receptor agonist is administered (added) to a slice of the hippocampus of a mammal to which an antidepressant has been administered. In such a case, the amount of the dopamine D1 receptor agonist or the dopamine D1 receptor antagonist is preferably 0.001 uM to 50 uM and more preferably 0.01 uM to 20 uM.

The step of detecting a greater increase in dopamine D1 receptor expression and/or dopamine D1 receptor signaling in comparison with the case where (A1) the antidepressant or (B2) a compound having the action of dopamine D1 receptor agonist is administered is preferably a step of detecting a greater increase in dopamine D1 receptor expression and/or dopamine D1 receptor signaling in a mammal to which (A1) an antidepressant and (B2) the compound having the action of dopamine D1 receptor agonist have been administered. Also, as a comparative control, a step of detecting a greater increase in dopamine D1 receptor expression and/or signaling in a mammal to which (A1) the antidepressant or (B2) the compound having the action of dopamine D1 receptor agonist has been administered may be performed. Such a comparative control step may be performed at the same time as Combined Administration Step 4, or before or after Combined Administration Step 4. In the case where the comparative step is performed before Combined Administration Step 4, the greater increase in the dopamine D1 receptor expression and/or of dopamine D1 receptor signaling may be detected and the value of expression level and/or signaling level may be determined beforehand.

The expression of a dopamine D1 receptor and the detection thereof, and the dopamine D1 receptor signaling and the detection thereof may be the same as described above.

“A greater increase in dopamine D1 receptor expression and/or dopamine D1 receptor signaling” means the following situation: the levels of the dopamine D1 receptor expression and/or dopamine D1 receptor signaling of (1) mammals to, which (A1) the antidepressant or (B2) a compound having the action of dopamine D1 receptor agonist has been administered, and (2) mammals to which (A1) an antidepressant and (B2) the compound having the action of dopamine D1 receptor agonist have been administered, are each averaged and statistically processed, and the comparison shows that the level of the dopamine D1 receptor expression and/or dopamine D1 receptor signaling of (2) is relatively higher than that of (1).

Regarding the greater increase in dopamine D1 receptor expression and/or dopamine D1 receptor signaling, the level of the dopamine D1 receptor expression and/or dopamine D1 receptor signaling of (2) mammals to which (A1) the antidepressant and (B2) the compound having the action of dopamine D1 receptor agonist have been administered, is higher than those of (1) mammals to which (A1) the antidepressant or a compound having the action of dopamine D1 receptor agonist has been administered, preferably by 1.1 to 100 times, more preferably by 1.5 to 20 times, and still more preferably by 2.0 to 10 times. The candidate medicines found by this screening are used for an animal experiment, a clinical application, or the like, through which an appropriate administration amount, an appropriate administration period, or the like can be determined depending on the targeted subject or the administration route.

The screening method of this embodiment may be performed one to several times. For example, the screening method of this embodiment may be performed in the same way except that the compound having the action of dopamine D1 receptor agonist used in the first screening is changed to another compound having the action of dopamine D1 receptor agonist in the second screening. In the case where the increase in the dopamine D1 receptor expression and/or dopamine D1 receptor signaling in the second screening is greater than the increase in the dopamine D1 receptor expression and/or dopamine D1 receptor signaling in the first screening, the compound having the action of dopamine D1 receptor agonist used in the second screening may be regarded as another candidate compound. Therefore, the screening method of this embodiment enables screening for a dopamine D1 receptor activating agent having a strong action of activating a dopamine D1 receptor, the agent being alternative to the compounds having the action of dopamine D1 receptor agonist (including a dopamine D1 receptor agonist). The same applies to the screening for a dopamine D1 receptor antagonist.

The present invention relates to a kit for screening for an antidepressant that in combination with a dopamine D1 receptor agonist or a dopamine D1 receptor antagonist provides an improvement in treatment of depression, the kit comprising either (B1) a dopamine D1 receptor agonist or (C1) a dopamine D1 receptor antagonist, and either or both of (D1) a detection reagent for a depression-related gene and (E1) a detection reagent for dopamine D1 receptor signaling. This kit may be used for the above described screening method. Examples of the combination of components in the kit of the present invention include (31), (D1), and (E1); (B1) and (D1); (B1) and (E1); (C1), (D1), and (E1); (C1) and (D1); and (C1) and (E1).

In the kit, (B1) the dopamine D1 receptor agonist in the kit may be the same as the above, but is not limited thereto. (D1) The detection reagent for a depression-related gene may be any as long as it detects the expression of the above depression-related gene etc., and publicly known detection reagents, such as a primer or an antibody, may be used. (E1) The detection reagent for dopamine D1 receptor signaling may be any as long as it detects phosphorylation or the like of, for example, DARPP-32 or ERK, and publicly known detection reagents, such as a phosphorylation state-specific antibody, may be used.

The present invention relates to a kit for screening for a dopamine D1 receptor agonist that in combination with an antidepressant provides an improvement in treatment of depression, the kit comprising (A1) an antidepressant, and either or both of (D1) a detection reagent for a depression-related gene and (E1) a detection reagent for dopamine D1 receptor signaling. This kit may be used for the above described screening method. Examples of the combination of components in the kit of the present invention include (A1), (D1), and (E1); (A1) and (D1); and (A1) and (E1).

In the kit, (A1) the antidepressant may be the same as the above, but is not limited thereto. (D1) The detection reagent for a depression-related gene may be any as long as it detects the expression of the above depression-related gene etc., and publicly known detection reagents, such as a primer or an antibody, may be used. (E1) The detection reagent for dopamine D1 receptor signaling may be any as long as it detects phosphorylation or the like of, for example, DARPP-32 or ERK, and publicly known detection reagents, such as a phosphorylation state-specific antibody, may be used.

### EXAMPLES

Hereinafter, the present invention will be illustrated by Examples, but it is not limited thereto.

### Example 1

### 1. Establishment of Fluoxetine Chronic Administration Model and Induction of Dematuration of Hippocampal Dentate Gyrus

Mice (sex: male, strain: C57Bl/6NCrS1c) were purchased from Japan SLC, and as a sustained-release antidepressant, a fluoxetine pellet (15 mg/(kg day)×14 days) as described below was subcutaneously implanted to each of the mice to prepare fluoxetine chronic administration mouse models. As the fluoxetine pellets, pellets capable of sustainably releasing fluoxetine (Sigma, #F132) were custom-made by Innovative Research of America. The method of subcutaneously implanting each pellet was as follows: a small incision about 5 mm was made in the neck skin of each mouse, a pellet was inserted through the incision to a depth of about 2 cm with tweezers for indwelling, and the skin was sutured.

After a fluoxetine pellet or a placebo pellet was subcutaneously implanted and 14 days of treatment was given, the gene expression in the hippocampal dentate gyrus was analyzed by RT-PCR. Total RNA was isolated from the hippocampal dentate gyrus of the mice treated with the fluoxetine pellet or the control pellet. First strand cDNA was prepared from 1 ug of DNaseI-treated total RNA using a reverse transcriptase QuantiTect (registered trademark, Qiagen, Valencia, Calif.). Gene expression was quantified using SYBR green (2×SYBR Green PCR Master Mix; Qiagen, Valencia, Calif.) according to the manufacturer's directions. Quantitative RT-PCR was performed using LightCycler (registered trademark) 480II real-time PCR detection system (Roche, Mannheim, Germany) in the following conditions (95° C.: 5 minutes; and then 40 cycles of 95° C.: 10 seconds, 60° C.: 30 seconds, and 65° C.: 1 minute). Beta-actin was amplified in all the samples for standardization of expression data. Whether or not the DNA region of each targeted gene was amplified was confirmed by melting curve analysis after RT-PCR (95° C.: 1 minute, 55° C.: 30 seconds, 95° C.: 30 seconds). Gene primers used in the quantitative real-time PCR are shown in Table 1. The Ct value used was the average of 2 RT-PCR results.

TABLE 1

| | Forward | Reverse |
|---|---|---|
| D1R | 5′-GCCGCTGTCATCAGGTTTC-3′ | 5′-GGCCAAAAGCCAGCAATCT-3′ |
| Calbindin | 5′-TCTGGCTTCATTTCGACGCTG-3′ | 5′-ACAAAGGATTTCATTTCCGGTGA-3′ |
| Desmoplakin | 5′-GCTGAAGAACACTCTAGCCCA-3′ | 5′-ACTGCTGTTTCCTCTGAGACA-3′ |
| Tryptophan 2,3-dioxygenase | 5′-ATGAGTGGGTGCCCGTTTG-3′ | 5′-GGCTCTGTTTACACCAGTTTGAG-3′ |
| Interleukin-1 receptor | 5′-GTGCTACTGGGGCTCATTTGT-3′ | 5′-GGAGTAAGAGGACACTTGCGAAT-3′ |
| Doublecortin | 5′-GCAATGGGGACCCCTTTTC-3′ | 5′-GGTGTAGATGTTCCTAACCGC-3′ |
| β-actin | 5′-AGTGTGACGTTGACATCCGTA-3′ | 5′-GCCAGAGCAGTAATCTCCTTCT-3′ |

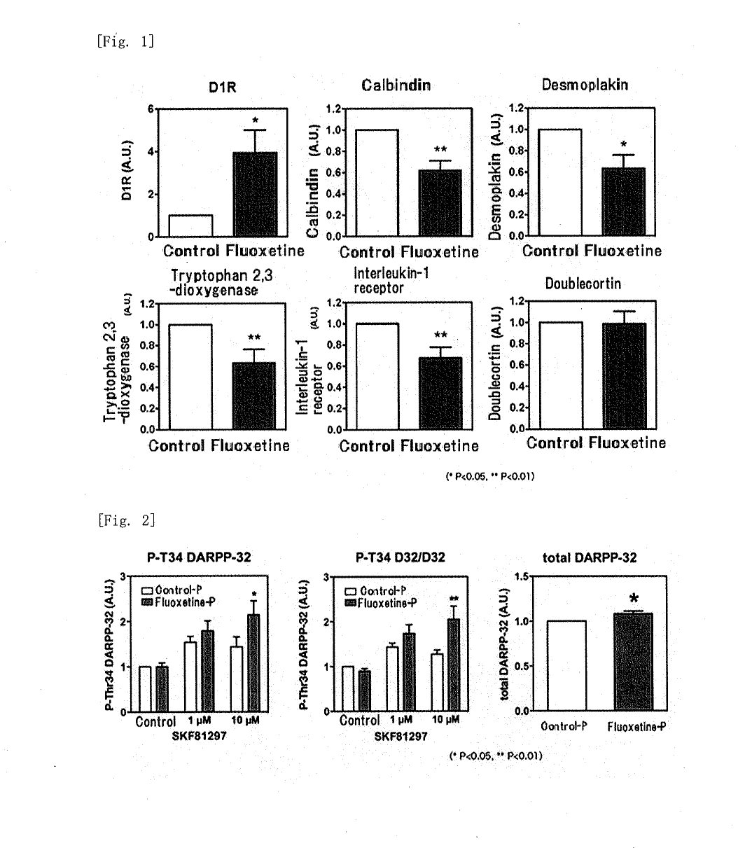

The mice treated with the fluoxetine pellet showed less mRNA expression of calbindin, desmoplakin, tryptophan 2,3-dioxygenase, and interleukin-1 receptor as compared to the mice treated with the placebo pellet (control), that is, showed the pattern of dematuration of hippocampal dentate gyrus. Further, increase in the mRNA expression of dopamine D1 receptor was observed. The results are shown in FIG. 1. These results are similar to those obtained in the method for administration in drinking water, where fluoxetine in the same amount as above was dissolved in water and administered for the same period as above.

### 2. Fluoxetine-Induced Increase in Expression of Dopamine D1 Receptor in Dematured Dentate Gyrus

The protein expression of dopamine D1 receptor in the hippocampal dentate gyrus of mice treated with a fluoxetine pellet or a control pellet was confirmed as follows.

After a fluoxetine pellet or a placebo pellet was subcutaneously implanted and 14 days of treatment was given, the protein expression of D1 receptor in the hippocampal dentate gyrus was analyzed by western blotting. The samples isolated from the hippocampal dentate gyrus of mice treated with a fluoxetine pellet or a control pellet were electrophoresed using acrylamide gel, and separated proteins were electrically transferred to a nitrocellulose membrane. The D1 receptor protein transferred to the membrane was immunologically detected using a D1 receptor antibody. In the immunological detection, Odyssey near-infrared fluorescent imaging system (LI-COR Biosciences, Lincoln, Nebr.) was used.

As a result, the hippocampal dentate gyrus of mice treated with a fluoxetine pellet showed 4 times higher protein expression of dopamine D1 receptor as compared to the mice treated with a placebo pellet.

### 3. Fluoxetine-Induced Increase in Dopamine D1 Receptor Signaling in Dematured Dentate Gyrus

Using slices of the hippocampal dentate gyrus of mice which were treated for 14 days with a subcutaneously implanted fluoxetine pellet, dopamine D1 receptor signaling was analyzed.

After a fluoxetine pellet or a placebo pellet was subcutaneously implanted and 14 days of treatment was given, slices of the hippocampal dentate gyrus were prepared. Harvested mouse brain was kept cool in ice-cooled and oxygenated Krebs-HCO₃⁻ buffer, and using vibratome VT1000S (Leica Microsystems, Nussloch, Germany), 350-micrometer-thick continuous coronal slices were prepared. From the coronal slices, slices of the hippocampal dentate gyrus were cut out and used for experiments. Each slice of the hippocampal dentate gyrus was preincubated in 2 mL of Krebs-HCO₃⁻ buffer for 60 minutes and treated with a dopamine D1 receptor agonist, namely SKF81297, and dopamine D1 receptor signaling was analyzed. As an indicator of dopamine D1 receptor signaling, phosphorylation of DARPP-32 (Thr34, PKA site) and of ERK was analyzed. The phosphorylation was analyzed by western blotting with a phosphorylation state-specific antibody.

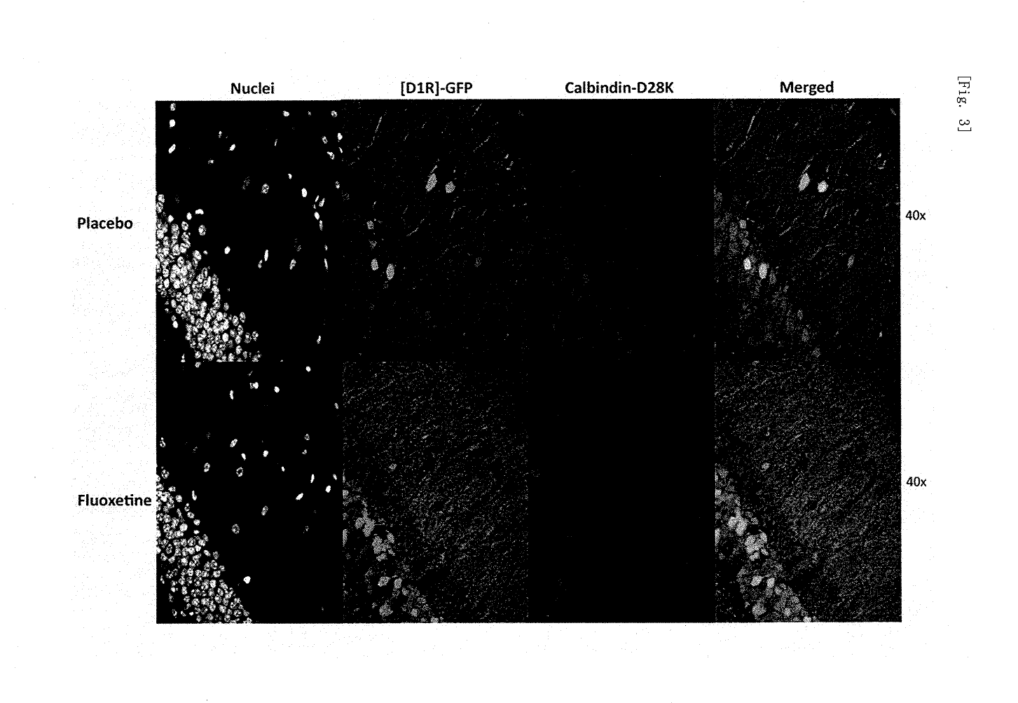

As a result, in the fluoxetine chronic administration mice, phosphorylation of DARPP-32 (Thr34, PKA site) and ERK induced by the dopamine D1 receptor agonist SKF81297 was increased. It was revealed that, in the fluoxetine-induced dematured dentate gyrus, dopamine D1 receptor signaling was doubled. The results are shown in FIG. 2.

### 4. Identification of Dopamine D1 Receptor Expressing Cells in Dematured Dentate Gyrus

For the purpose of identifying dopamine D1 receptor expressing cells in the fluoxetine-induced dematured dentate gyrus, immunohistochemical analysis was performed using the dentate gyrus tissue of dopamine D1 receptor-GFP mice (Tg(Drd1a-EGFP) X60Gsat/Mmmh from GENSAT Project at Rockefeller University) into which a placebo pellet or a fluoxetine pellet had been implanted.

Each mouse was anesthetized with sodium pentobarbital and perfused with 4% paraformaldehyde (PFA) in 0.1 M phosphate buffered saline (PBS). The brain was removed and subjected to immersion fixation in the same fixing fluid at 4° C. for another 2 hours and 14-mm-thick frontal-plane sections were prepared using a cryostat (Leica). The sections were washed with tris buffered saline (pH 7.4) containing Tween 20. For immunostaining, frozen sections were incubated with the following primary antibody: rabbit anti-calbindin D28K polyclonal antibody (1:3000 dilution; SWANT) at 4° C. for 18 hours. The anti-calbindin D28K polyclonal antibody is a maturation marker for granule cells. For detection of antigen localization, the sections were incubated with Alexa Fluor (594)-conjugate goat anti-rabbit IgG (1:400 dilution; Invitrogen) at 4° C. for 2 hours. Further, for nuclear staining, the sections were stained with DAPI. Fluorescent signals were each analyzed using a confocal laser scanning microscope (LSM5 Pascal, Zeiss).

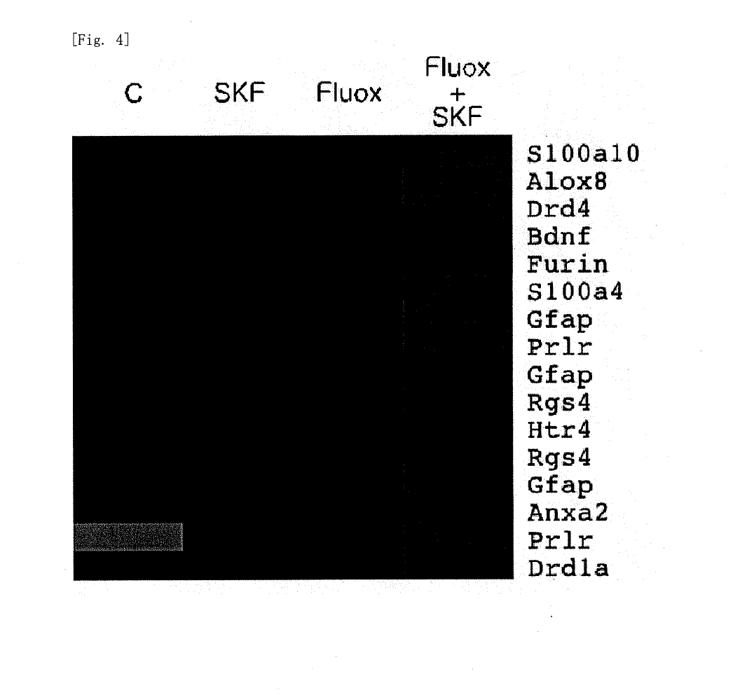

The expression of dopamine D1 receptor-GFP was observed in granule cells expressing a neural marker (NeuN) for granular cell layer (not shown in FIG. 3). However, the expression of dopamine D1 receptor-GFP hardly corresponded to those of a neural stem cell marker (Ki67) or an immature neural marker (doublecortin, or calretinin) (not shown in FIG. 3). Interestingly, in the dentate gyrus of the mice treated with a placebo pellet, granule cells where calbindin was highly expressed, namely matured granule cells, poorly expressed dopamine D1 receptor-GFP while granule cells poorly expressing calbindin, namely dematured granule cells, expressed dopamine D1 receptor-GFP. Meanwhile, in the fluoxetine-induced dematured dentate gyrus, granule cells where calbindin was poorly expressed showed significantly increased expression of dopamine D1 receptor-GFP (FIG. 3). Therefore, it was revealed that fluoxetine chronic administration induces dematuration of the existing mature granule cells, and that the dematured granular cells show an increased expression of dopamine D1 receptor. The results are shown in Table 3.

### 5. Function Analysis of Dopamine D1 Receptor Expressed in Dematured Dentate Gyrus

In order to analyze the functional role of dopamine D1 receptor expressed in the fluoxetine-induced dematured dentate gyrus, to placebo pellet treatment mice (control) or fluoxetine pellet treatment mice (15 mg/(kg day)×14 days), a dopamine D1 receptor agonist SKF81297 (3 mg/(kg day) i.p.×5 days) or physiological saline was administered for 5 consecutive days from the 10th day to the 14th day from the start of the fluoxetine treatment. That is, control mice (placebo pellet treatment+physiological saline administration), dopamine D1 receptor agonist sole administration mice (placebo pellet treatment+SKF81297 administration), antidepressant sole administration mice (fluoxetine pellet treatment+physiological saline administration), and antidepressant and dopamine D1 receptor agonist combined administration mice (fluoxetine pellet treatment+SKF81297 administration) were prepared. Then, microarray analysis of genes expressed in the dentate gyrus of these mice was performed.

The microarray analysis was performed as follows. Total RNA was isolated from the hippocampus of each mouse by TRIzol method (Invitrogen, Carlsbad, Calif.) and then purified using an RNeasy column (Qiagen, Valencia, Calif.). Double strand cDNA was synthesized from the total RNA, and in vitro transcription reaction was performed using biotin-labeled RNA prepared from the cDNA. The labeled RNA was hybridized with Mouse Genome 430 2.0 Array (Affymetrix, Santa Clara, Calif.) comprising specific probe sets, and washing was performed according to the manufacturer's recommendations. The hybridized probe array was stained with streptoavidin conjugate phycoerythrin, and each GeneChip was scanned using Affymetrix GeneChip scanner 3000 (GCS3000). GeneChip analysis was performed using Microarray Analysis Suite version 5.0. All the genes displayed on the GeneChip were comprehensively standardized. For each probe, 2-way analysis of variance was performed, and probes showing a significantly changed expression level were identified.

As a result, while neither the SKF81297 sole administration nor the fluoxetine sole administration affected the expression of genes, the combined administration of SKF81297 with fluoxetine significantly enhanced the expression of depression-related gene group, which was expected to be increased by fluoxetine. Gene expression of the depression-related genes, such as D1R, p11 (S100A10), annexin A2, tissue plasminogen activator, BDNF, and the like, was increased. The results of microarray analysis are shown in FIG. 4, and measured values of the mRNA expression levels corresponding to the microarray analysis results are shown in Table 2. In addition, the enhancement of the mRNA expression of dopamine D1 receptor and BDNF, and the inhibition of the mRNA expression of calbindin were confirmed.

TABLE 2

| P_Sal | P_SKF | F_Sal | F_SKF | |
|---|---|---|---|---|
| C | D1R | Fluox | Fluox + D1R | GeneSymbol |
| 461.56 | 469.21 | 455.84 | 1304.89 | S100a10 |
| 2.02 | 2.12 | 2.11 | 375.92 | Alox8 |
| 29.28 | 30.92 | 35.57 | 218.01 | Drd4 |
| 1312.91 | 1516.05 | 1495.90 | 4479.96 | Bdnf |
| 742.58 | 837.03 | 799.17 | 1530.94 | Furin |
| 337.31 | 389.62 | 353.74 | 1309.39 | S100a4 |
| 115.89 | 140.79 | 116.30 | 629.97 | Gfap |
| 48.03 | 61.94 | 48.69 | 232.52 | Prir |
| 3008.11 | 4062.15 | 3185.26 | 11142.08 | Gfap |
| 2347.43 | 2742.21 | 3659.58 | 10283.28 | Rgs4 |
| 723.76 | 852.76 | 903.85 | 1754.81 | Htr4 |
| 687.50 | 1053.99 | 1215.07 | 3276.89 | Rgs4 |
| 1308.81 | 1962.44 | 1757.42 | 5555.81 | Gfap |
| 483.27 | 684.27 | 568.07 | 1362.01 | Anxa2 |
| 36.03 | 160.90 | 84.69 | 1100.83 | Prir |
| 46.26 | 43.87 | 114.21 | 326.47 | Drd1a |

Therefore, it was revealed that fluoxetine, which is an antidepressant (SSRI), induces dematuration of the mouse hippocampal dentate gyrus and stimulates the dematured granule cells to express dopamine D1 receptor. Further, it was revealed that the dopamine D1 receptor expressed in the granule cells, by an interaction with fluoxetine, significantly enhances gene expression in the dentate gyrus. The results suggest that medicines targeting dopamine D1 receptor may be useful as a combined medicine for treatment of depression, the medicine enhancing the action of an antidepressant (FIG. 5), and therefore the present study developed from “Study of intermediate phenotype of psychiatric disorder model mouse” is a very important study that contributes to the development of medicines for depression treatment.

### Example 2

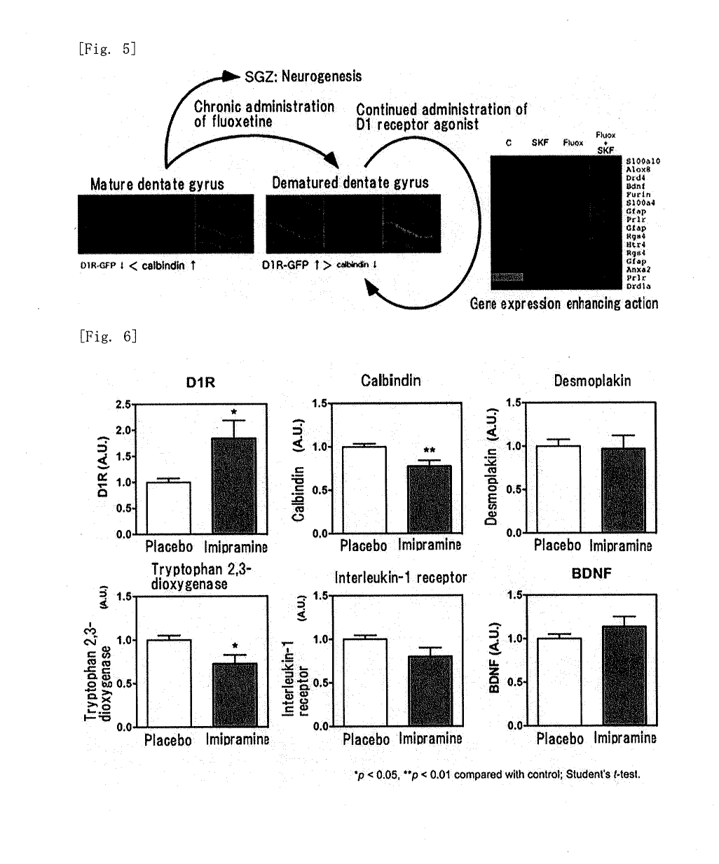

Experiments were conducted in the same manner as in “1. Establishment of fluoxetine chronic administration model and induction of dematuration of hippocampal dentate gyrus” in Example 1 except that imipramine was used instead of fluoxetine. The mice treated with the imipramine pellet showed less mRNA expression of calbindin, desmoplakin, tryptophan 2,3-dioxygenase, and interleukin-1 receptor as compared to the mice treated with the placebo pellet (control), that is, showed the pattern of dematuration of hippocampal dentate gyrus. Further, increase in the mRNA expression of dopamine D1 acceptor was observed. The results are shown in FIG. 6.

### Example 3

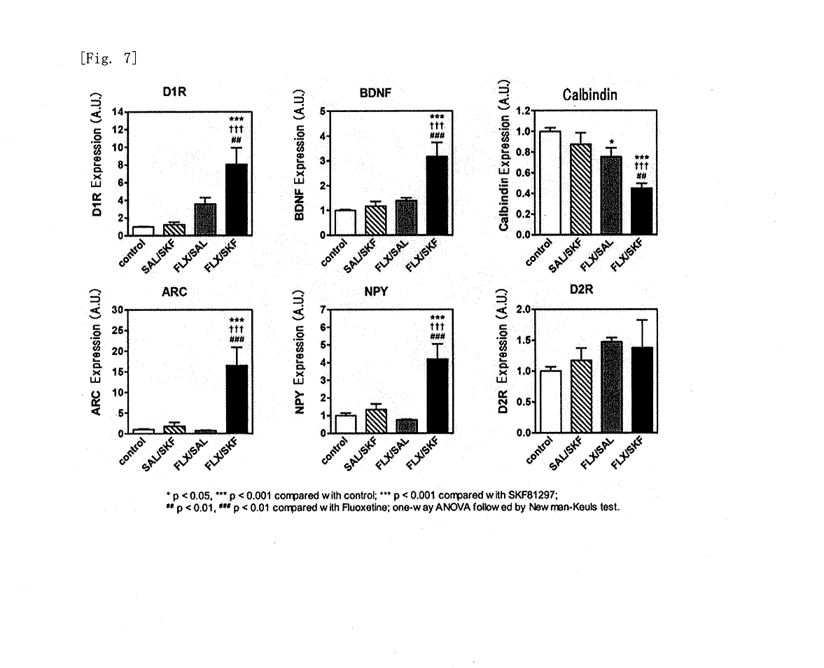

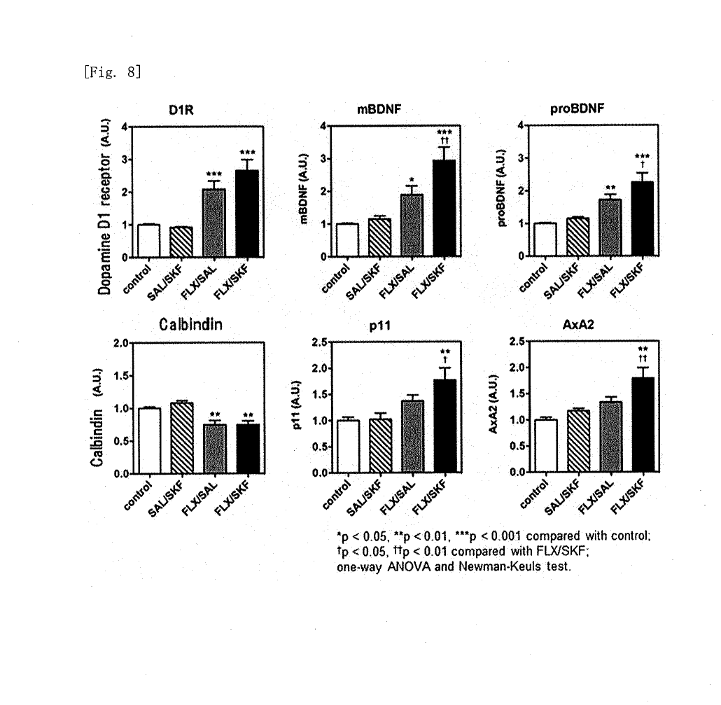

In the same manner as in Example 1, a dopamine D1 receptor agonist, namely SKF81297 was administered to the chronic fluoxetine administration model mice in Example 1, and mRNA expression in the hippocampal dentate gyrus was examined. The results are shown in FIG. 7. FIG. 7 clearly shows the beneficial effects of the present invention on the depression-related genes. Further, the protein expression level was measured. The results are shown in FIG. 8. As shown in FIG. 8, it was revealed that, regarding the protein level as well, combination use of SKF81297 further increases the depression-related gene expression increased by fluoxetine. These results clearly show the beneficial effects of the present invention. It was also confirmed that, by the use of the method of the present invention, a dopamine D1 receptor agonist that, in combination with an antidepressant, improves the treatment of depression can be obtained.

The present invention is not limited to the embodiments and examples described above. Various modifications can be made within the scope of technical ideas of the invention, and embodiments obtainable by combining technical means disclosed in different embodiments are also included in the technical scope of the present invention. All the academic literature and patent literature described in this description are incorporated herein by reference.

### INDUSTRIAL APPLICABILITY

According to the present invention, combined use of (A1) an antidepressant with either (B1) a dopamine D1 receptor agonist or (C1) a dopamine D1 receptor antagonist enhances the effect of the antidepressant and improves the problems of the antidepressant. Specifically, rapid action of the antidepressant, improvement in the improvement rate and the cure rate of depression, improvement in the symptoms of depression in refractory depression, and inhibition of recurrence of depression, can be expected.

1. A combination medicine for treatment of depression, comprising a combination of (A1) an antidepressant and either (B1) a dopamine D1 receptor agonist or (C1) a dopamine D1 receptor antagonist.

2. The combination medicine according to claim 1, wherein combined use of (A1) the antidepressant and (B1) the dopamine D1 receptor agonist enhances an increase in dopamine D1 receptor signaling and in depression-related gene expression.

3. The combination medicine according to claim 1, wherein (A1) the antidepressant is a sustained-release preparation.

4. The combination medicine according to claim 1, wherein (A1) the antidepressant is (a1) a tricyclic antidepressant, (a2) a tetracyclic antidepressant, (a3) a selective serotonin reuptake inhibitor, or (a4) a selective serotonin and/or noradrenaline reuptake inhibitor.

5. The combination medicine according to claim 1, wherein the selective serotonin reuptake inhibitor is fluoxetine, fluvoxamine, sertraline, paroxetine, or escitalopram.

6. The combination medicine according to claim 1, wherein (B1) the dopamine D1 receptor agonist is SKF81297, SKF83959 or SKF38393.

7. A pharmaceutical composition, comprising a combination of (A1) an antidepressant and either (B1) a dopamine D1 receptor agonist or (C1) the dopamine D1 receptor antagonist.

8. A method for treatment of depression, comprising the steps of: administering a therapeutically effective amount of (A1) an antidepressant to a mammal, and administering a therapeutically effective amount of (B1) a dopamine D1 receptor agonist or (C1) a dopamine D1 receptor antagonist to the mammal simultaneously with or after the start of the administration of the antidepressant.

9. A method for administering an antidepressant and either a dopamine D1 receptor agonist or a dopamine D1 receptor antagonist for treatment of depression, the method comprising the steps of: administering a therapeutically effective amount of (A1) an antidepressant to a mammal, and administering a therapeutically effective amount of either (B1) a dopamine D1 receptor agonist or (C1) a dopamine D1 receptor antagonist to the mammal simultaneously with or after the start of the administration of the antidepressant.

10. The method according to claim 9, wherein the antidepressant is administered in the form of a sustained-release preparation.

11. Use of (A1) an antidepressant and either (B1) a dopamine D1 receptor agonist or (C1) a dopamine D1 receptor antagonist for the manufacture of medicine for treatment of depression.

12. A method for screening for an antidepressant that in combination with a dopamine D1 receptor agonist provides an improvement in treatment of depression, the method comprising the steps of: administering, to a mammal, (A2) a compound having an antidepressant action and (B1) a dopamine D1 receptor agonist, and detecting a greater increase in depression-related gene expression in comparison with the case where (A2) the compound having an antidepressant action or (B1) the dopamine D1 receptor agonist is solely administered.

13. The method according to claim 12, wherein the administration of (A2) the compound having an antidepressant action and (B1) the dopamine D1 receptor agonist shows an increase in depression-related gene expression greater than the increase shown by sole administration of (A2) the compound having an antidepressant action and greater than the increase shown by sole administration of (B1) the dopamine D1 receptor agonist.

14. The method according to claim 12, wherein the depression-related gene is any one or more selected from the group consisting of dopamine D1 receptor, p11, Annexin A2, tissue plasminogen activator, ARC, neuropeptide Y and BDNF.

15. The method according to claim 12, wherein the depression-related gene expression is detected based on mRNA or protein expression of the depression-related gene.

16. A method for screening for a dopamine D1 receptor agonist that in combination with an antidepressant provides an improvement in treatment of depression, the method comprising the steps of: administering, to a mammal, (A1) an antidepressant and (B2) compound having a dopamine D1 receptor activating action, and detecting a greater increase in depression-related gene expression in comparison with the case where (A1) the antidepressant or (B2) the compound having a dopamine D1 receptor activating action is solely administered.

17. A method for screening for an antidepressant that in combination with a dopamine D1 receptor agonist provides an improvement in treatment of depression, the method comprising the steps of: administering, to a mammal, (A2) a compound having an antidepressant action and (B1) a dopamine D1 receptor agonist, and detecting a greater increase in dopamine D1 receptor expression and/or dopamine D1 receptor signaling in comparison with the case where (A2) the compound having an antidepressant action or (B1) the dopamine D1 receptor agonist is administered.

18. The method according to claim 17, wherein the administration of (A2) the compound having an antidepressant action and (B1) the dopamine D1 receptor agonist shows a greater increase in dopamine D1 receptor expression and/or dopamine D1 receptor signaling than the increase shown by the administration of (A2) the compound having an antidepressant action or (B1) the dopamine D1 receptor agonist.

19. The method according to claim 17, wherein the dopamine D1 receptor expression is detected based on mRNA or protein expression of dopamine D1 receptor.

20. The method according to claim 17, wherein the dopamine D1 receptor signaling is detected based on phosphorylation of DARPP-32 or ERK.

21. A method for screening for a dopamine D1 receptor agonist that in combination with an antidepressant provides an improvement in treatment of depression, the method comprising the steps of: administering, to a mammal, (A1) an antidepressant and (B2) a compound having a dopamine D1 receptor activating action, and detecting a greater increase in dopamine D1 receptor expression and/or dopamine D1 receptor signaling in comparison with the case where (A1) the antidepressant or (B2) the compound having a dopamine D1 receptor activating action is administered.

22. A kit for screening for an antidepressant that in combination with either a dopamine D1 receptor agonist or a dopamine D1 receptor antagonist provides an improvement in treatment of depression, the kit comprising either (B1) a dopamine D1 receptor agonist or (C1) a dopamine D1 receptor antagonist, and either or both of (D1) a detection reagent for a depression-related gene and (E1) a detection reagent for dopamine D1 receptor signaling.

23. A kit for screening for a dopamine D1 receptor agonist that in combination with an antidepressant provides an improvement in treatment of depression, the kit comprising (A1) an antidepressant, and either or both of (D1) a detection reagent for a depression-related gene and (E1) a detection reagent for dopamine D1 receptor signaling.
